(19) United States
(12) Patent Application Publication
(10) Pub. No.: US 2009/0149533 A1
(43) Pub. Date: Jun. 11, 2009

(54) NOVEL FENOFIBRATE FORMULATIONS AND RELATED METHODS OF TREATMENT

(75) Inventors: Orn Almarsson, Shrewsbury, MA (US); Pasut Ratanabanangkoon, Bangkok (TH); Julius Remenar, Framingham, MA (US); Hector Guzman, Jamaica Plain, MA (US)

Correspondence Address: RatnerPrestia, P.O. Box 980, Valley Forge, PA, 19482-0980, US

(73) Assignee: TransForm Pharmaceuticals, Inc., Lexington, MA (US)

(21) Appl. No.: 11/573,237

(22) PCT Filed: Aug. 4, 2005
(86) PCT No.: PCT/US05/27806

§ 371 Date: Aug. 1, 2008

Related U.S. Application Data
(60) Provisional application No. 60/599,320, filed on Aug. 6, 2004.
(60) Provisional application No. 60/604,861, filed on Aug. 27, 2004.
(60) Provisional application No. 60/655,762, filed on Feb. 24, 2005.
(60) Provisional application No. 60/687,704, filed on Jun. 6, 2005.

(51) Int. Cl. A61K 31/216; C07C 69/76; A61P 29/00

(52) U.S. Cl. 514/512; 560/8

(57) ABSTRACT

The invention provides novel omega-3 ester-based oil solutions of fenofibrate. These solutions are substantially free of any food effect, effective in small volumes, and readily bioavailable. Notably, because the solutions of the invention contain an omega-3 ester-based oil as the major ingredient, they not only provide an antihyperlipidemic effect due to the fenofibrate active ingredient, they also provide recommended daily dosages of omega-3 oils (i.e., approximately 1 gram of omega-3 oil per day), or a portion thereof.

### FIELD OF THE INVENTION

The invention provides novel omega-3 ester-based oil liquid formulations of fenofibrate. These solutions are substantially free of food effect, effective in small volumes, and readily bioavailable.

The invention also provides novel fenofibrate formulations in which fenofibrate is dissolved in a vehicle comprising an omega-3 ester-based oil, an alcohol, and a surfactant.

### BACKGROUND OF THE INVENTION

The fibrates (fibric acid derivatives) include clofibrate (ATROMID-S®), fenofibrate (TRICOR®), bezafibrate (BEZALIP®), ciprofibrate, beclofibrate, etofibrate, and gemfibrozil (LOPID®). Fibrates act as prodrugs and are metabolized in vivo to species that are active in the treatment of hyperlipidemia. Fibrates are known to be peroxisome proliferator-activated receptor alpha (PPARα) agonists.

Fenofibrate (2-[4-(4-chlorobenzoyl)phenoxy]-2-methyl-propanoic acid 1-methylethyl ester) is a benzophenone which contains a para-chlorophenyl group and a para-isopropyloxycarbonylisopropoxyphenyl group, both of which are substantially hydrophobic groups. Fenofibrate is practically insoluble in water. It is normally poorly and variably absorbed in the fasted state and currently is prescribed to be taken with food.

Fenofibrate is absorbed and then hydrolyzed by tissue and plasma esterases to fenofibric acid, a fibrate-active species which has an elimination half-life of approximately twenty hours. Fenofibric acid lowers plasma triglycerides by potentially inhibiting triglyceride synthesis, leading to a reduction of the amount of triglyceride-rich lipoprotein (VLDL) released into the circulation. Fenofibric acid also stimulates the catabolism of VLDL and reduces serum uric acid levels in hyperuricemic and normal individuals by increasing the urinary excretion of uric acid. Measurement of the detected amount of fenofibric acid in the blood of a patient can reflect the efficacy of fenofibrate uptake.

Patient uptake of a fibrate such as fenofibrate is affected by food, i.e., fenofibrate exhibits a “positive food effect”. A positive food effect exists when the amount of an active drug taken into the blood from a given oral dosage form by a fasting patient is less than the amount of the active drug taken into the blood from the same dosage form by the same patient who has eaten a particular type of meal around the time of drug administration. A negative food effect exists when the amount of an active drug taken into the blood from a given oral dosage form by a fasting patient is more than the amount of the active drug taken into the blood from the same dosage form by the same patient who has eaten a particular type of meal around the time of drug administration.

Known fenofibrate dosage forms include Tricor® micronized tablets in which fenofibrate powder is co-micronized with a solid wetting agent such as sodium lauryl sulfate. The co-micronized powder is mixed with excipients such as lactose, starch, cross-linked polyvinyl pyrrolidone (PVP), and magnesium stearate.

U.S. Pat. No. 6,667,064 discloses compositions for treating hypertriglyceridemia which comprise fibrates and a mixture of fatty acyl compounds that have a polyunsaturated fatty acid content of at least sixty-five weight percent and which include γ-linoleic acid, α-linolenic acid, and stearidonic acid.

The hypotriglyceridemic effects of omega-3 oils from fish oils are well established. Amounts both above and below about 1 gram per day of omega-3 oils from fish oil have been shown to decrease serum triglyceride concentrations by about 25% to about 40%, decrease VLDL blood plasma levels, and to increase both LDL and HDL plasma levels (See e.g., Harris, William S, Clin. Cardiol. 22, (Suppl. II), II-40-II-43 (1999)). A dose-response relationship exists between omega-3 oil intake and triglyceride lowering. Postprandial triglyceridemia is especially sensitive to chronic omega-3 oil consumption. Kris-Etherton, et al., Circulation. 2002; 106:2747.

While there are numerous known fenofibrate dosage forms, the need continues to exist for commercially practicable fenofibrate formulations that exhibit enhanced bioavailability, are readily formulated and administered, and comprise ingredients that enhance the VLDL-lowering effect of fenofibrate.

Ideally, such formulations would not exhibit any food effect, thereby providing health care providers and patients with a wide latitude in selecting convenient and effective antihyperlipidemia dosage regimens.

Additionally, it would prove advantageous, both clinically and economically, to minimize the size or volume of such a fenofibrate dosage form and to ensure formulation homogeneity. It would also prove advantageous to increase the solubility of fenofibrate in liquid formulations.

### SUMMARY OF THE INVENTION

The invention provides novel omega-3 ester-based oil liquid formulations of fenofibrate having unexpected properties. These formulations are unexpectedly effective in small volumes (due to unexpectedly concentrated formulations) and readily bioavailable. Notably, because the formulations of the invention contain an omega-3 ester-based oil as the major ingredient, they not only provide an antihyperlipidemic effect due to the fenofibrate active ingredient, they also provide recommended daily dosages of omega-3 oils (i.e., one gram of omega-3 oil per day, as per AHA guidelines), or a portion thereof.

The invention also provides novel liquid fenofibrate formulations in which fenofibrate is dissolved in a vehicle comprising an omega-3 ester-based oil, a C₁ to C₄ alcohol, and a surfactant. Surprisingly, such formulations help to increase solubility of fenofibrate in the non-diluted state.

In one embodiment, liquid formulations of the invention comprise fenofibrate dissolved in a liquid vehicle at a concentration of at least about 50, 60, 70, 80, 90, 100, 110, 120, 130, 140, 150, 160, 170, 180, 190, 200, 210, 220, 230, 240, 250, 260, 270, 280, 290, or 300 milligrams of fenofibrate per milliliter of formulation. This active ingredient concentration is surprisingly achieved, in part, by adding a C₁ to C₄ alcohol to the formulation. An increase in the length of the unsaturated omega-3 carbon chain also results in increased solubility of fenofibrate. Additionally, the use of a monoalkyl ester (e.g., ethyl ester) proves surprisingly advantageous to the solubility value. In several formulations of the invention, the molar ratio of unsaturated moieties contained within the omega-3 ester-based oil to the total moles of omega-3 ester-based oil is about 3 to about 6, for example about 3, 4, 5, or 6.

Because of their homogeneity, high potency, and minimal effective volumes, formulations of the invention can be administered in a dosage form consisting of one or two capsules as defined hereinafter and at least about 400, 450, 500, 600, 700, 800, 900, or 1000 mg per capsule or per dose of an omega-3 oil.

In one embodiment, formulations of the invention comprise an omega-3 alkyl ester, such as an omega-3 ethyl ester. In another embodiment, formulations of the invention comprise an omega-3 mono-, di-, or triglyceride oil.

In another embodiment, about 5.00, 6.00, 7.00, 8.00, 9.00, 10.00, 11.00, 12.00, 13.00, 14.00, 15.00, 16.00, 17.00, 18.00, 19.00, or 20.00% by volume of an alcohol (for example, ethanol) is included in formulations of the invention to enhance the solubility of fenofibrate in the omega-3 ester-based oil.

In another embodiment, a medium-chain triglyceride such as a caprylic/capric triglyceride (e.g., Neobee® M5 Stepan Company) or a medium chain mono-diglyceride such as caprylic/capric mono-diglyceride (e.g., Capmul® MCM, Abitec Corporation) may be included in a formulation of the invention to facilitate digestion of the formulation or reduce the food effect. In another embodiment, a surfactant may be included in a formulation of the invention to enhance digestion of the formulation or reduce the food effect.

In another embodiment, the invention provides a liquid formulation comprising about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by weight of an omega-3 ester or omega-3 alkyl ester, about 5.00, 6.00, 7.00, 8.00, 9.00, 10.00, 11.00, 12.00, 13.00, 14.00, 15.00, 16.00, 17.00, 18.00, 19.00, or 20.00% by weight of an alcohol, and about 5.00, 6.00, 7.00, 8.00, 9.00, 10.00, 11.00, 12.00, 13.00, 14.00, 15.00, 16.00, 17.00, 18.00, 19.00, or 20.00% by weight of fenofibrate. In another embodiment, the formulation comprises about 75.00, 76.00, 77.00, 78.00, 79.00, or 80.00% by weight of an omega-3 ester or omega-3 alkyl ester, about 5.00, 6.00, 7.00, 8.00, 9.00, 10.00, 11.00, 12.00, 13.00, 14.00, 15.00, 16.00, 17.00, 18.00, 19.00, or 20.00% by weight of an alcohol, and about 6.00, 7.00, 8.00, 9.00, 10.00, 11.00, or 12.00% by weight of fenofibrate.

In another embodiment, the invention provides a liquid formulation consisting of about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by weight of an omega-3 ester or omega-3 alkyl ester, about 5.00, 6.00, 7.00, 8.00, 9.00, 10.00, 11.00, 12.00, 13.00, 14.00, 15.00, 16.00, 17.00, 18.00, 19.00, or 20.00% by weight of an alcohol, about 5.00, 6.00, 7.00, 8.00, 9.00, 10.00, 11.00, 12.00, 13.00, 14.00, or 15.00% by weight of a medium chain triglyceride or a mono-diglyceride, and about 5.00, 6.00, 7.00, 8.00, 9.00, 10.00, 11.00, 12.00, 13.00, 14.00, 15.00, 16.00, 17.00, 18.00, 19.00, or 20.00% by weight of fenofibrate.

In another embodiment, the invention provides a liquid formulation comprising about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by weight of an omega-3 ester or omega-3 alkyl ester, about 5.00, 6.00, 7.00, 8.00, 9.00, 10.00, 11.00, 12.00, 13.00, 14.00, 15.00, 16.00, 17.00, 18.00, 19.00, or 20.00% by weight of an alcohol, about 5.00, 6.00, 7.00, 8.00, 9.00, 10.00, 11.00, 12.00, 13.00, 14.00, 15.00, 16.00, 17.00, 18.00, 19.00, 20.00, 21.00, 22.00, 23.00, 24.00, or 25.00% by weight of a surfactant, and about 5.00, 6.00, 7.00, 8.00, 9.00, 10.00, 11.00, 12.00, 13.00, 14.00, 15.00, 16.00, 17.00, 18.00, 19.00, or 20.00% by weight of fenofibrate.

In another embodiment, the invention provides a liquid formulation comprising less than or equal to about 10.00, 11.00, 12.00, 13.00, 14.00, 15.00, 16.00, 17.00, 18.00, 19.00, 20.00, 21.00, 22.00, 23.00, 24.00 or 25.00% surfactant.

In another embodiment, the invention provides a formulation comprising about 75.00, 76.00, 77.00, 78.00, 79.00, or 80.00% by weight of an omega-3 ethyl ester with greater than or equal to about 90.00 percent purity, about 5.00, 6.00, 7.00, 8.00, 9.00, 10.00, 11.00, 12.00, 13.00, 14.00, 15.00, 16.00, 17.00, 18.00, 19.00, or 20.00% by weight of ethanol, and about 6.00, 7.00, 8.00, 9.00, 10.00, 11.00, or 12.00% by weight of fenofibrate. In another embodiment, the invention provides a formulation comprising about 75.00, 76.00, 77.00, 78.00, 79.00, or 80.00% by weight of an omega-3 ethyl ester with a composition greater than or equal to about 90.00 percent EPA and DHA, about 5.00, 6.00, 7.00, 8.00, 9.00, 10.00, 11.00, 12.00, 13.00, 14.00, 15.00, 16.00, 17.00, 18.00, 19.00, or 20.00% by weight of ethanol, and about 6.00, 7.00, 8.00, 9.00, 10.00, 11.00, or 12.00% by weight of fenofibrate. In another embodiment, the ethanol may be replaced with glycerol. In another embodiment, the ethanol may be replaced with a mixture of ethanol and glycerol.

In another embodiment, the purity of omega-3 esters or omega-3 alkyl esters is at least about 50.00 percent by weight, at least about 60.00 percent by weight, at least about 70.00 percent by weight, at least about 75.00 percent by weight, at least about 80.00 percent by weight, or at least about 85.00 percent by weight. In another embodiment, the purity of omega-3 esters or omega-3 alkyl esters is about 25.00, 30.00, 35.00, 40.00, 45.00, 50.00, 55.00, 60.00, 65.00, 70.00, 75.00, 80.00, 85.00, 90.00, 95.00, 99.00 percent or more by weight. In another embodiment, the purity of omega-3 esters or omega-3 alkyl esters is between about 25.00 and about 100.00 percent by weight, between about 40.00 and about 100.00 percent by weight, between about 50.00 and about 100.00 percent by weight, between about 60.00 and about 100.00 percent by weight, between about 70.00 and about 100.00 percent by weight, between about 75.00 and about 100.00 percent by weight, between about 75.00 and about 95.00 percent by weight, between about 75.00 and about 90.00 percent by weight, or between about 80.00 and about 85.00 percent by weight. In another embodiment, the purity of omega-3 esters or omega-3 alkyl esters is about 100.00 percent by weight, about 99.00 percent by weight, at least about 96.00 percent by weight, at least about 92.00 percent by weight, at least about 90.00 percent by weight, at least about 85.00 percent by weight, at least about 80.00 percent by weight, at least about 75.00 percent by weight, at least about 70.00 percent by weight, at least about 65.00 percent by weight, at least about 60.00 percent by weight, at least about 55.00 percent by weight, or at least about 50.00 percent by weight.

In another embodiment, the oil composition comprising EPA and DHA is at least about 50.00 percent by weight, at least about 60.00 percent by weight, at least about 70.00 percent by weight, at least about 75.00 percent by weight, at least about 80.00 percent by weight, or at least about 84.00 percent by weight of EPA and DHA. In another embodiment, the oil composition comprising EPA and DHA is about 25.00, 30.00, 35.00, 40.00, 45.00, 50.00, 55.00, 60.00, 65.00, 70.00, 75.00, 80.00, 85.00, 90.00, or 95.00 percent by weight of EPA and DHA. In another embodiment, the oil composition comprising EPA and DHA is between about 25.00 and about 95.00 percent by weight, between about 40.00 and about 95.00 percent by weight, between about 50.00 and about 95.00 percent by weight, between about 60.00 and about 95.00 percent by weight, between about 70.00 and about 95.00 percent by weight, between about 75.00 and about 95.00 percent by weight, between about 75.00 and about 90.00 percent by weight, between about 75.00 and about 85.00 percent by weight, or between about 80.00 and about 85.00 percent by weight of EPA and DHA. In another embodiment, the oil composition comprising EPA and DHA is about 99.00 percent by weight, about 96.00 percent by weight, about 92.00 percent by weight, about 90.00 percent by weight, about 84.00 percent by weight, about 80.00 percent by weight, about 75.00 percent by weight, about 70.00 percent by weight, about 65.00 percent by weight, about 60.00 percent by weight, about 55.00 percent by weight, or about 50.00 percent by weight of EPA and DHA.

In another embodiment, the omega-3 ester or omega-3 alkyl ester has about a 23:19 ratio of EPA:DHA, about a 75:11 ratio of EPA:DHA, about a 95:1 ratio of EPA:DHA, about a 9:2 ratio of EPA:DHA, about a 10:1 ratio of EPA:DHA, about a 5:1 ratio of EPA:DHA, about a 3:1 ratio of EPA:DHA, about a 2:1 ratio of EPA:DHA, about a 1:1 ratio of EPA:DHA, about a 1:2 ratio of EPA:DHA, about a 1:3 ratio of EPA:DHA, or about a 1:5 ratio of EPA:DHA. In another embodiment, the omega-3 ester or omega-3 alkyl ester has about a 95:1 ratio of EPA:DHA, about a 75:1 ratio of EPA:DHA, about a 50:1 ratio of EPA:DHA, about a 25:1 ratio of EPA:DHA, about a 20:1 ratio of EPA:DHA, about a 15:1 ratio of EPA:DHA, about a 10:1 ratio of EPA:DHA, about a 7.5:1 ratio of EPA:DHA, about a 5:1 ratio of EPA:DHA, about a 4:1 ratio of EPA:DHA, about a 3:1 ratio of EPA:DHA, about a 2:1 ratio of EPA:DHA, about a 1.5:1 ratio of EPA:DHA, about a 1:1 ratio of EPA:DHA, about a 1:1.5 ratio of EPA:DHA, about a 1:2 ratio of EPA:DHA, about a 1:3 ratio of EPA:DHA, or about a 1:5 ratio of EPA:DHA. In another embodiment, the omega-3 ester or omega-3 alkyl ester has from about a 95:1 ratio to about a 1:5 ratio of EPA:DHA, from about a 50:1 ratio to about a 1:1 ratio of EPA:DHA, from about a 25:1 ratio to about a 1:1 ratio of EPA:DHA, from about a 10:1 ratio to about a 1:1 ratio of EPA:DHA, from about a 5:1 ratio to about a 1:1 ratio of EPA:DHA, from about a 3:1 ratio to about a 1:1 ratio of EPA:DHA, from about a 2:1 ratio to about a 1:1 ratio of EPA:DHA, or from about a 1.5:1 ratio to about a 1:1 ratio of EPA:DHA. In another embodiment, the omega-3 ester or omega-3 alkyl ester has at least about a 1:5 ratio of EPA:DHA, at least about a 1:1 ratio of EPA:DHA, at least about a 1.5:1 ratio of EPA:DHA, at least about a 2:1 ratio of EPA:DHA, at least about a 3:1 ratio of EPA:DHA, at least about a 5:1 ratio of EPA:DHA, or at least about a 10:1 ratio of EPA:DHA.

In another embodiment, a liquid formulation of the invention comprises about 50, 60, 70, 80, 90, 100, 110, 120, 130, 140, 150, 160, 170, 180, 190, or about 200 milligrams of fenofibrate dissolved in a vehicle comprising an omega-3 ethyl ester, about 5.00, 6.00, 7.00, 8.00, 9.00, 10.00, 11.00, 12.00, 13.00, 14.00, 15.00, 16.00, 17.00, 18.00, 19.00, or 20.00% by volume of ethanol, and a medium-chain triglyceride or a mono-diglyceride, wherein the formulation of the composition on a weight percentage basis is as follows: about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by weight of the omega-3 ethyl ester, about 5.00, 6.00, 7.00, 8.00, 9.00, 10.00, 11.00, 12.00, 13.00, 14.00, 15.00, 16.00, 17.00, 18.00, 19.00, or 20.00% by weight of ethanol, about 5.00, 6.00, 7.00, 8.00, 9.00, 10.00, 11.00, 12.00, 13.00, 14.00, or 15.00% by weight of the medium chain triglyceride or mono-diglyceride, and about 5.00, 6.00, 7.00, 8.00, 9.00, 10.00, 11.00, 12.00, 13.00, 14.00, 15.00, 16.00, 17.00, 18.00, 19.00, or 20.00% by weight of fenofibrate.

In another embodiment, a liquid formulation of the invention comprises fenofibrate dissolved in a vehicle at a concentration of about 50, 60, 70, 80, 90, 100, 110, 120, 130, 140, 150, 160, 170, 180, 190, or 200 milligrams of fenofibrate per milliliter of vehicle, wherein the vehicle consists of an omega-3 ethyl ester, about 5.00, 6.00, 7.00, 8.00, 9.00, 10.00, 11.00, 12.00, 13.00, 14.00, 15.00, 16.00, 17.00, 18.00, 19.00, 20.00, 21.00, 22.00, 23.00, 24.00, 25.00, 26.00, 27.00, 28.00, 29.00, or 30.00% by volume of ethanol, and a medium-chain triglyceride or mono-diglyceride, and wherein: (1) the formulation composition on a weight percentage basis is as follows: about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by weight of the omega-3 ethyl ester, about 5.00, 6.00, 7.00, 8.00, 9.00, 10.00, 11.00, 12.00, 13.00, 14.00, 15.00, 16.00, 17.00, 18.00, 19.00, or 20.00% by weight of ethanol, about 5.00, 6.00, 7.00, 8.00, 9.00, 10.00, 11.00, 12.00, 13.00, 14.00, or 15.00% by weight of the medium chain triglyceride or mono-diglyceride, and about 5.00, 6.00, 7.00, 8.00, 9.00, 10.00, 11.00, 12.00, 13.00, 14.00, 15.00, 16.00, 17.00, 18.00, 19.00, or 20.00% by weight of fenofibrate, and (2) the molar ratio of unsaturated moieties contained with the omega-3 ester-based oil to the total moles of omega-3 ester-based oil is about 3 to about 6.

In another embodiment, a capsule dosage form of the invention comprises fenofibrate dissolved in a vehicle at a concentration of about 50, 60, 70, 80, 90, 100, 110, 120, 130, 140, 150, 160, 170, 180, 190, or 200 milligrams of fenofibrate per milliliter of vehicle, wherein the vehicle comprises an omega-3 ethyl ester, about 5.00, 6.00, 7.00, 8.00, 9.00, 10.00, 11.00, 12.00, 13.00, 14.00, 15.00, 16.00, 17.00, 18.00, 19.00, 20.00, 21.00, 22.00, 23.00, 24.00, 25.00, 26.00, 27.00, 28.00, 29.00, or 30.00% by volume of ethanol, and either a medium-chain triglyceride or mono-diglyceride, and wherein the composition of the formulation on a weight percentage basis is as follows: about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by weight of the omega-3 ethyl ester, about 5.00, 6.00, 7.00, 8.00, 9.00, 10.00, 11.00, 12.00, 13.00, 14.00, 15.00, 16.00, 17.00, 18.00, 19.00, or 20.00% by weight of ethanol, about 5.00, 6.00, 7.00, 8.00, 9.00, 10.00, 11.00, 12.00, 13.00, 14.00, or 15.00% by weight of the medium chain triglyceride or mono-diglyceride, and about 5.00, 6.00, 7.00, 8.00, 9.00, 10.00, 11.00, 12.00, 13.00, 14.00, 15.00, 16.00, 17.00, 18.00, 19.00, or 20.00% by weight of fenofibrate, and optionally, wherein the liquid formulation is encapsulated in an enteric coating as defined hereinafter.

In another embodiment, a liquid formulation of the invention comprises a mixture of fenofibrate dissolved in a vehicle comprising an omega-3 ester or omega-3 alkyl ester and a C₁ to C₄ alcohol, wherein:

(a) the formulation comprises (i) about 5.00, 6.00, 7.00, 8.00, 9.00, 10.00, 11.00, 12.00, 13.00, 14.00, 15.00, 16.00, 17.00, 18.00, 19.00, or 20.00% by weight of fenofibrate (ii) about 55.00, 56.00, 57.00, 58.00, 59.00, 60.00, 61.00, 62.00, 63.00, 64.00, 65.00, 66.00, 67.00, 68.00, 69.00, 70.00, 71.00, 72.00, 73.00, 74.00, 75.00, 76.00, 77.00, 78.00, 79.00, or 80.00% by weight of an omega-3 ester or omega-3 alkyl ester (iii) about 5.00, 6.00, 7.00, 8.00, 9.00, 10.00, 11.00, 12.00, 13.00, 14.00, 15.00, 16.00, 17.00, 18.00, 19.00, or 20.00% by weight of a C₁ to C₄ alcohol, and (iv) about 5.00, 6.00, 7.00, 8.00, 9.00, 10.00, 11.00, 12.00, 13.00, 14.00, 15.00, 16.00, 17.00, 18.00, 19.00, 20.00, 21.00, 22.00, 23.00, 24.00, or 25.00% by weight of a surfactant; and
(b) the solubility of the fenofibrate in the vehicle is about 50, 60, 70, 80, 90, 100, 110, 120, 130, 140, 150, 160, 170, 180, 190, 200, 210, 220, 230, 240, 250, 260, 270, 280, 290, or 300 milligrams per milliliter at 25 degrees C.

In an alternative embodiment, the surfactant adds solubilization power to the undiluted non-aqueous formulation.

In another embodiment, a liquid formulation of the invention comprises a mixture of fenofibrate dissolved in a vehicle comprising an omega-3 ester or omega-3 alkyl ester and a C₁ to C₄ alcohol, wherein:

(a) the formulation comprises (i) about 5.00, 6.00, 7.00, 8.00, 9.00, 10.00, 11.00, 12.00, 13.00, 14.00, 15.00, 16.00, 17.00, 18.00, 19.00, or 20.00% by weight of fenofibrate (ii) about 55.00, 56.00, 57.00, 58.00, 59.00, 60.00, 61.00, 62.00, 63.00, 64.00, 65.00, 66.00, 67.00, 68.00, 69.00, 70.00, 71.00, 72.00, 73.00, 74.00, 75.00, 76.00, 77.00, 78.00, 79.00, or 80.00% by weight of an omega-3 ester or omega-3 alkyl ester (iii) about 5.00, 6.00, 7.00, 8.00, 9.00, 10.00, 11.00, 12.00, 13.00, 14.00, 15.00, 16.00, 17.00, 18.00, 19.00, or 20.00% by weight of a C₁ to C₄ alcohol, and (iv) about 5.00, 6.00, 7.00, 8.00, 9.00, 10.00, 11.00, 12.00, 13.00, 14.00, 15.00, 16.00, 17.00, 18.00, 19.00, 20.00, 21.00, 22.00, 23.00, 24.00, or 25.00% by weight of a surfactant.

In another embodiment, the surfactant is not present within the mixture of fenofibrate dissolved in a vehicle comprising an omega-3 ester or omega-3 alkyl ester and a C₁ to C₄ alcohol. In another embodiment, the surfactant is present only within the gelatin shell of a dosage form.

In an alternative embodiment, the surfactant increases the solubility of the fenofibrate in the undiluted non-aqueous formulation. In another alternative embodiment, the surfactant increases in vivo bioavailability in the fasted state.

In another embodiment, the present invention provides a method for increasing the solubility of fenofibrate in an omega-3 oil by adding an alcohol, such as a C₁ to C₄ alcohol.

In another embodiment, the present invention provides a novel polymorph of fenofibrate.

In another embodiment, the present invention provides a method of making a polymorph of fenofibrate, comprising:
(a) combining fenofibrate with one or more components so as to form a solution of fenofibrate;
(b) decreasing the temperature of said solution; and
(c) collecting a precipitated solid.

The invention provides novel surfactant-containing and surfactant-free, omega-3 ester-based oil liquid medicaments of fenofibrate. These medicaments are effective in small volumes (due to unexpectedly concentrated liquid medicaments) and readily bioavailable. Notably, because the medicaments of the invention contain an omega-3 ester-based oil as the major ingredient, they not only provide an antihyperlipidemic effect due to the fenofibrate active ingredient, they also provide recommended daily dosages of omega-3 oils (i.e., one gram of omega-3 oil per day, as per AHA guidelines), or a portion thereof.

The invention also provides novel liquid fenofibrate medicaments in which fenofibrate is dissolved in a vehicle comprising an omega-3 ester-based oil, a C₁ to C₄ alcohol, and a surfactant. Surprisingly, such medicaments provide increased fenofibrate solubility in the non-diluted state.

These and other embodiments are described in greater detail in the following detailed description.

### BRIEF DESCRIPTION OF THE FIGURES

FIG. 1 illustrates the solubility of fenofibrate in E463808-ethanol solutions at 25° C.

FIG. 2 illustrates the effect of temperature on fenofibrate solubility in pure E463808 and E463808-ethanol solutions.

FIG. 3 illustrates the Van't Hoff temperature dependence of fenofibrate solubility in pure E463808 and in E463808-ethanol mixtures.

FIGS. 4A and 4B illustrate polarized light microscopy images at room temperature and 45° C. of fenofibrate crystals dispersed in an enteric coating comprising polymeric matrices of Eudragit® L100 and ethanol.

FIGS. 5A and 5B illustrate powder X-ray diffraction (PXRD) diffractograms of fenofibrate crystals dispersed in polymeric matrices comprising Eudragit® L100 and ethanol. FIGS. 5C, 5D, and 5E show PXRD diffractograms, of the fenofibrate-Eudragit® L100 globule, a fenofibrate crystal from the globule, and fenofibrate (Form I) powder, respectively.

FIG. 6 illustrates the digestion of fenofibrate formulations in various media.

FIG. 7 illustrates the solubility of fenofibrate in E463808-additive mixtures at 25° C.

FIG. 8 shows a PXRD diffractogram of a fenofibrate polymorph (Form II).

FIG. 9 shows a semi-log plot of the mean plasma concentration of fenofibric acid in humans following oral administration.

FIG. 10 shows fenofibrate solubility as a function of ethanol concentration.

FIG. 11 shows fenofibrate solubility as a function of temperature.

### DETAILED DESCRIPTION OF THE INVENTION

As used herein, the following terms have the following respective meanings.

“Alkyl” means a straight chain or branched, saturated or unsaturated alkyl, cyclic or non-cyclic hydrocarbon having from 1 to 10 carbon atoms. Representative saturated straight chain alkyls include methyl, ethyl, n-propyl, n-butyl, n-pentyl, n-hexyl, and the like; while saturated branched alkyls include isopropyl, sec-butyl, isobutyl, tert-butyl, isopentyl, and the like. Unsaturated alkyls contain at least one double or triple bond between adjacent carbon atoms (also referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1 butynyl, and the like. Representative saturated cyclic alkyls include cyclopropyl, cyclobutyl, cyclopentyl, cyclohexyl, and the like; while unsaturated cyclic alkyls include cyclopentenyl and cyclohexenyl, and the like. Cycloalkyls are also referred to herein as “carbocyclic” rings systems, and include bi- and tri-cyclic ring systems having from 8 to 14 carbon atoms such as a cycloalkyl (such as cyclopentane or cyclohexane) fused to one or more aromatic (such as phenyl) or non-aromatic (such as cyclohexane) carbocyclic rings. “Alkenyl” can be used in the context of omega-3 formulations to refer to unsaturation.

As used herein, the term “adjunctively administered” refers to the administration of one or more compounds or active ingredients in addition to a pharmaceutically acceptable salt, solvate, co-crystal, or polymorph of a racemate or stereoisomer of fenofibrate, such as fenofibrate, either simultaneously with the same or at intervals prior to, during, or following administration of the pharmaceutically acceptable salt, solvate, or polymorph of a racemate or stereoisomer of fenofibrate, such as fenofibrate, to achieve the desired therapeutic or prophylactic effect.

“Fatty acids” are an important component of nutrition. Fatty acids (also described as “free acids” or “free fatty acids”) are carboxylic acids and are classified based on the length and saturation characteristics of the carbon chain. Short chain fatty acids have 2 to about 5 carbons and are typically saturated. Medium chain fatty acids have from about 6 to about 14 carbons and are also typically saturated. Long chain fatty acids have from about 15 to 24 or more carbons and may also be saturated or unsaturated. In longer fatty acids there may be one or more points of unsaturation, giving rise to the terms “monounsaturated” and “polyunsaturated”, respectively. Long chain polyunsaturated fatty acids (LCPs or LC-PUFAs) having 20 or more carbons are used in the instant invention.

“Long chain” mono-, di-, tri-glycerides, esters, tatty acids, etc. are defined as having about 15, 16, 17, 18, 19, 20, 21, 22, 23, 24 or more carbons and may also be saturated or unsaturated. “Medium chain” mono-, di-, tri-glycerides, esters, fatty acids, etc. are defined as having about 6, 7, 8, 9, 10, 11, 12, 13, or 14 carbons and may also be saturated or unsaturated. “Short chain” mono-, di-, tri-glycerides, esters, fatty acids, etc. are defined as having about 2, 3, 4, or 5, carbons and may also be saturated or unsaturated.

“Mono-diglyceride” and “mono-diglycerides” refer to a mixture or mixtures comprising both monoglycerides and diglycerides. A non-limiting example of a mono-diglyceride is Capmul® MCM, which comprises a mixture of caprylic and capric fatty acids in the form of monoglycerides and diglycerides. Certain mixtures of monoglycerides and diglycerides may be specifically stated as mono-diglycerides according to the present invention. Mono-diglycerides can comprise other species such as, for example, triglycerides and glycerol.

“C₁ to C₄ alcohols” include, but are not limited to, methanol, ethanol, propanol, butanol, isopropanol, isobutanol, tert-butanol, glycerol, and propylene glycol.

Several liquid formulations of the present invention comprise alcohol. The term “alcohol” may be used to describe any —OH bearing hydrocarbon. Hydrocarbons bearing 2, 3, 4, 5, 6, or more —OH groups are also included by the term “alcohol.” According to the invention, alcohols comprise those —OH bearing hydrocarbons with 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more carbon atoms.

LC-PUFAs are categorized according to the number and position of double bonds in the fatty acids according to an accepted nomenclature that is well-known to those of ordinary skill in the art. There are two series or families of LC-PUFAs, depending on the position of the double bond closest to the methyl end of the fatty acid: the n-3 series contains a double bond at the third carbon, while the n-6 series has no double bond until the sixth carbon. Thus, arachidonic acid (AA or ARA) has a chain length of 20 carbons and 4 double bonds beginning at the sixth carbon. As a result, it is referred to as “20:4 n-6”. Similarly, docosahexaenoic acid (DHA) has a chain length of 22 carbons with 6 double bonds beginning with the third carbon from the methyl end and is thus designated “22:6 n-3”. Another important LC-PUFA is eicosapentaenoic acid (EPA) which is designated (20:5 n-3). The terms “n-3” and “omega-3” are used interchangeably.

The biosynthetic pathways for AA (n-6 series) and DHA (n-3 series) from their respective C18 precursors are distinct, but share elongation and desaturation steps and are well understood. Thus, other important LC-PUFAs are the C18 fatty acids that are precursors in these biosynthetic pathways, for example, linoleic (18:2 n-6) and gamma-linolenic (18:3 n-6) acids in the n-6 pathway, and alpha-linolenic (18:3 n-3) and stearidonic (18:4 n-3) acids in the n-3 pathway.

Fatty acids are often found in nature as acyl radicals esterified to alcohols. A glyceride is such an ester of one or more fatty acids with glycerol (1,2,3-propanetriol). If only one position of the glycerol backbone molecule is esterified with a fatty acid, a “monoglyceride” is produced; if two positions are esterified, a “diglyceride” is produced; and if all three positions of the glycerol are esterified with fatty acid a “triglyceride” or “triacylglycerol” is produced. A glyceride is called “simple” if all esterified positions contain the same fatty acid; or “mixed” if different fatty acids are involved. A phospholipid is a special type of diglyceride, wherein the third position on the glycerol backbone is bonded to a nitrogen containing compound such as choline, serine, ethanolamine, inositol, etc., via a phosphate ester. Triglycerides and phospholipids are often classified as long chain (from about 15 to 24 or more carbons) or medium chain (from about 6 to about 14 carbon), according to the fatty acids attached thereto.

Typically commercially available monoglycerides contain varying amounts of di- and triglycerides in addition to their monoglyceride content. For example, a monoglyceride (e.g., Akoline, by Karlshamns AB, Sweden) can comprise about 50-65% monoglyceride, about 25-35% diglyceride, and up to about 5% triglycerides.

The “essential fatty acids” (EFAs) are of two types, the n-3 (or omega-3) series derived from alpha-linolenic acid and the n-6 (or omega-6) series derived from linoleic acid.

An “omega-3 fatty acid” is a n-3 polyunsaturated long-chain fatty acids (n-3 PUFA) and is defined to include any carboxylic acid having at least 15 carbon atoms and having at least 3 non-conjugated cis-unsaturated bonds, the distal one of which from the methyl end of the fatty acid chain being located between the third and fourth carbon atoms. The omega-3 fatty acids therefore include C₁₆-C₂₄ alkanoic acids comprising 5-7 double bonds, wherein the last double bond is located between the third and fourth carbon atom from the methyl end of the fatty acid chain.

Examples of omega-3 fatty acids include stearidonic acid (SDA, C18:4), eicosatetraenoic acid (ETA, C20:4), eicosapentaenoic acid (EPA, C20:5), docosapentaenoic acid (DPA, C22:5), and docosahexaenoic acid (DHA, C22:6). For the purpose of the invention, alpha-linolenic acid (ALA, C18:3) is considered an omega-3 fatty acid. Terms such as “EPA” and “DHA” denote species of omega-3 oil and do not describe whether such oils exist as, for example, triglycerides, diglycerides, monoglycerides, free acids, esters, or salts.

Omega-3 fatty acids include synthetic or naturally occurring omega-3 fatty acids, such as those found in fish oil, e.g., marine mammal fat, cod liver oil, walnuts and walnut oil, wheat germ oil, rapeseed oil, soybean lecithin, soybeans, tofu, common beans, butternuts, seaweed and flax seed oil. An omega-3 fatty acid may also be derived from genetically engineered sources such as transgenic plants. See, e.g., Frasier, et al., Nat Biotechnol. 2004 May 16.

An “omega-3 oil” or “omega-3” is any oil comprising a source of omega-3 fatty acids, omega-3 esters, omega-3 alkyl esters, or omega-3 mono-, di-, or triglycerides, such as fish oil, e.g., marine mammal fat, cod liver oil, walnuts and walnut oil, wheat germ oil, rapeseed oil, soybean lecithin derived oils, soybean derived oils, tofu derived oils, common bean derived oils, butternut derived oils, seaweed derived oils, flax-borage oil, and flax seed oil. The Epax® (Pronova Biocare AS) brand of omega-3 oils are exemplary. Other omega-3 oils which can be used in making formulations of the invention include, but are not limited to, the omega-3 oil marketed under the tradename Omegabrite® (Omega Natural Science) and Epanova™ (Tillotts Pharma AG). Certain mixtures of esters, fatty acids, and/or mono- di-triglycerides may be specifically stated as oils according to the present invention. For example, a mixture consisting of omega-3 esters and fatty acids may be considered an omega-3 oil according to the present invention. In addition, one or more components may be specifically excluded from an omega-3 oil according to the present invention. For example, an omega-3 oil may specifically exclude esters, fatty acids, and/or mono- di-triglycerides according to the present invention. As such, a composition consisting of omega-3 esters, for example, is an omega-3 oil according to the present invention.

An “omega-3 alkyl ester” may be formed by transesterification of an omega-3 oil and an alcohol and either an acid or reducing agent. Generally, for the formation of lower alkyl esters, the alcohol is a lower alkyl alcohol containing from 1 to 6 carbon atoms (such as methanol or ethanol). In one embodiment, the alcohol is methanol (which reacts with glycerides to form methyl esters of the fatty acid residues) or ethanol (which reacts with glycerides to form ethyl esters of the fatty acid residues). In another embodiment, the alcohol is ethanol.

Liquid formulations and medicaments may be described as mixtures of two or more components “by volume,” which is herein defined as the volume due to one component divided by the volume of all components of the formulation. This ratio may be converted to or reported as a percentage of the total formulation volume. Such a quantity may also be indicated by “v/v” or “percent v/v.” Similarly, the phrases “by weight” and “by mass” describe the weight or mass due to one component divided by the weight or mass of all components of the formulation. This ratio may be converted to or reported as a percentage of the total formulation weight or mass. Such a quantity may also be indicated by “w/w”, “mass percent,” or “percent w/w.”

The term “E463808” is used to described an omega-3 oil which has a composition comprising 46% EPA, 38% DHA, and 8% other omega-3 oils (mass percent) where the EPA, DHA, and other omega-3 oils are ethyl esters.

The term “E107104” is used to describe an omega-3 oil which has a composition comprising 9.7% EPA, 71.4% DHA, and about 3.9% other omega-3 oils (mass percent) where the EPA, DHA, and other omega-3 oils are ethyl esters.

The term “E970002” is used to describe an omega-3 oil which has a composition comprising 97% EPA and about 2% other omega-3 oils (mass percent) where the EPA and other omega-3 oils are ethyl esters.

The term “TG361724” is used to describe an omega-3 oil which has a composition comprising 36% EPA (expressed as mass percent of free fatty acids), 17% DHA (expressed as mass percent of free fatty acids), and about 24% other omega-3 oils (mass percent) where the EPA, DHA, and other omega-3 oils are triglycerides.

The term “E351923” is used to describe an omega-3 oil which has a composition comprising 35% EPA (expressed as mass percent of free fatty acids), 19% DHA (expressed as mass percent of free fatty acids), and about 23% other omega-3 oils (mass percent) where the EPA, DHA, and other omega-3 oils are ethyl esters.

The term “E681010” is used to describe an omega-3 oil which has a composition comprising 67.8 percent EPA (mg/g), 9.9 percent DHA (mg/g), and about 9.6 percent other omega-3 oils (mg/g), where the EPA, DHA, and other omega-3 oils are ethyl esters.

“Fibrate” as used herein includes clofibrate (ATROMID-S®), fenofibrate (Tricor®), bezafibrate (BEZALIP®), ciprofibrate, beclofibrate, etofibrate, and gemfibrozil (LOPID®). “Fibrate” as defined herein also includes any composition or combination of compositions that contain a fibric acid or which generate a fibric acid in vivo as a metabolite (e.g., fenofibric acid).

The terms “an effective amount”, “therapeutic effective amount”, or “therapeutically effective amount” shall mean an amount or concentration of a composition according to the present invention which is effective in producing a desired result within the context of its administration or use, including, for example, lowering blood plasma triglyceride levels and in providing recommended dietary levels of omega-3 oil. Thus, the term “effective amount” is used throughout the specification to describe concentrations or amounts of formulations according to the present invention which may be used to produce a favorable change in the disease or condition treated, whether that change is a reduction in blood plasma triglyceride levels, an increase in blood plasma LDL levels, or other favorable physiological result.

The term “patient” includes an animal, mammal, or a human.

“Enteric coating” refers to a means for protecting acid unstable medication from the attack of the gastric fluid. Many enteric coatings can rapidly release the active drug in the proximal part of the gastrointestinal canal. Many enteric coatings are known to those skilled in the art including, as non-limiting examples, coatings comprised of an anionic polymer of methacrylic acid and methacrylates comprising a carboxyl group. For example, Eudragit® L100 (Rohm Pharma) can be used as an enteric coating.

A “liquid formulation” refers to a mixture wherein the majority of the API (active pharmaceutical ingredient) is in solution at equilibrium. For example, at least about 55.00, 60.00, 65.00, 70.00, 75.00, 80.00, 85.00, 90.00, 95.00, 96.00, 97.00, 98.00, 99.00, 99.50, or 99.99 percent of the fenofibrate in the liquid formulation is present in solution at equilibrium. Liquid formulations include, but are not limited to, semi-solid formulations.

The terms “physically stable” or “physical stability” refer to a liquid formulation of an API at equilibrium in which no crystals are present.

The terms “chemically stable” or “chemical stability” refer to a liquid formulation where there is a ≦3.0 percent loss of fenofibrate potency (recovered fenofibrate content) after 2 years at 25 degrees C.

“Surfactants” and “a surfactant of the invention” refer to a surface active compound which can alter the surface tension of a liquid in which it is dissolved and includes, but is not limited to, polyoxyl 20 stearate, polyoxyl 35 castor oil, poloxamers, polyoxyethylene sorbitan monoisostearate, polyethylene glycol 40 sorbitan diisostearate, polyoxyl 40 hydrogenated castor oil, polysorbate, polysorbate 20, polysorbate 40, polyoxyl 60 stearate, polysorbate 85, polysorbate 60, poloxamer 331, polyoxyethylene fatty acid esters, polyoxyl 40 castor oil, poloxamer 188, polyoxyethylene polyoxypropylene 1800, oleic acid, sodium desoxycholate, sodium lauryl sulfate, sorbitan monolaurate, sorbitan monooleate, sorbitan monopalmitate, sorbitan trioleate, N-carbamoyl methoxypolyethylene glycol 2000-1,2-distearol, myristic acid, steareth, polyoxyl 40 stearate, sucrose stearate, tocopherol, polyoxyl castor oil, triglyceride synthetic, trimyristin, tristearin, magnesium stearate, lecithin, lauryl sulfate, vitamin E, egg yolk phosphatides, docusate sodium, polysorbate 80, dimyristoyl phosphatidylglycerol, dimyristoyl lecithin, Capryol 90 (propylene glycol monocaprylate), Capryol PGMC (propylene glycol monocaprylate), deoxycholate, cholesterol, Cremophor RH, Cremophor EL, propylene glycol alginate, Croval A-10 (PEG 60 almond glycerides), Labrafil 1944 (oleoyl macrogol-6 glycerides), Labrafil 2125 (linoleoyl macrogol-6 glycerides), Labrasol (caprylocaproyl macrogol-8 glycerides), Lauroglycol 90 (propylene glycol monolaurate), Lauroglycol FCC (propylene glycol laurate), calcium stearate, Lecithin Centromix E, Lecithin Centrophase 152, Lecithin Centrol 3F21B, POE 26 glycerin, Olepal isosteariques (PEG-6 isostearate), Plurol diisostearique (polyglycerol-3-diisostearate), Plurol Oleique CC, POE 20 Sorbitan trioleate, Tagat TO (polyoxyethylene glycerol trioleate), or Solutol (Macrogol-15 hydroxystearate).

Surfactants also include, but are not limited to, polyoxyethylene 20 sorbitan monoleate, polyoxyethylene alkyl ethers of the Brig- or Volpo series, polyoxyethylene sorbitant fatty acid esters of the Tween- or Crillet series, polyoxyethylene stearates of the Cerosynt- or Myrj series, lecithin, poloxamers, d-alpha-tocopheryl polyethylene glycol 1000 succinate (Vitamin E TPGS, TPGS), saturated polyglycolized glycerides (Labrasol, Labrafil and Gelucires), cholic acid and salts of cholic acid, deoxycholic acid and salts of deoxycholic acid, taurocholic acid, salts of taurocholic acid, glycocholic acid, polyvinylpyrrolidone, cocamines, glyceryl stearates, glyceryl oleates, hydrogenated lanolins, lanolins, laurates and oleates, sorbitan laurates, sorbitan palmitates, sorbitan stearates, quaternium surfactants, sodium sulfates, glyceryl compounds, palmitic acid and its derivatives and oleic acid and its derivatives.

PEG-containing surfactants include, but are not limited to, Tween 85®, Tween 80®, and Cremophor® EL.

Acid-catalyzed transesterification may be carried out, for example, by incubating a triglyceride at from about 0° C. to about 150° C. in a mixture containing the alcohol and an acid (e.g., HCl), optionally under a non-oxidizing atmosphere and in the absence of water. In one embodiment, the triglyceride/acid/alcohol mixture is refluxed for at least about 2 hours. In another embodiment, the triglyceride/acid/alcohol mixture is maintained at from about 0° C. to about 50° C. overnight. Methanol may be used to form methyl esters, and ethanol may be used to form ethyl esters. Because acid-catalyzed transesterification is typically reversible, the alcohol commonly is present in a large excess so that the reaction proceeds essentially to completion. Often, the triglyceride concentration in the alcohol/acid mixture is from about 0.1 to about 15% by weight, or about 3% by weight. If the acid is HCl, the concentration of HCl in the alcohol/HCl mixture usually is from about 4 to about 15% by weight, or about 10% by weight. Such a mixture may be prepared by various methods known in the art, such as bubbling dry gaseous hydrogen chloride into dry ethanol, or adding 1 mL of acetylchloride to each 10 mL of alcohol (to form approximately 10% by weight HCl in alcohol).

Although HCl is common, other acids may alternatively be used. One such acid is sulfuric acid, which typically is used at a concentration of from about 0.5 to about 5% by weight in the alcohol. It should be noted, however, that because sulfuric acid is a strong oxidizing agent, it preferably is not used with long reflux times (i.e., greater than about 6 hours), at high concentrations (i.e., greater than about 5% by weight), or at high temperatures (i.e., greater than 150° C.). Another example of a suitable acid is boron trifluoride, which preferably is used at a concentration of from about 1 to about 20% by weight in the alcohol. Boron trifluoride, however, is less preferred than HCl because boron trifluoride has a greater tendency to produce undesirable byproducts.

In base-catalyzed transesterification, the omega-3 oil is transesterified by an alcohol in the presence of a basic catalyst. In this instance, the base may be, for example, sodium methoxide, potassium methoxide, elemental sodium, sodium hydroxide, or potassium hydroxide. Frequently, the volumetric ratio of omega-3 oil to the base/alcohol mixture is at least about 1:1, and commonly about 1:2. The concentration of the base in the alcohol preferably is from about 0.1 to about 2 M. The base-catalyzed transesterification reaction can be conducted at room temperature (i.e., at a temperature of from about 20° to about 25° C.) for from about 6 to about 20 hours. Alternatively, the base-catalyzed transesterification reaction is conducted at a temperature greater than room temperature.

The glyceride/alcohol/catalyst solution preferably is heated to a temperature of at least about 40° C., more preferably from about 70 to about 150° C., and most preferably at about 100° C. The solution can be heated using a reflux condenser so that the reaction mixture may be heated to temperatures above the boiling point of one or more components in the mixture without losing the components into the vapor phase (i.e., when the components vaporize, they rise into the reflux condenser which has a cooler temperature, thereby causing the vapor to condense into a liquid and flow back into the liquid mixture).

During the transesterification reaction, the reacting mixture is preferably placed under a non-oxidizing atmosphere, such as an atmosphere consisting essentially of a noble gas, N₂, or a combination thereof. Use of such an atmosphere is particularly preferred if the transesterification reaction is conducted over a period of time exceeding about 10 minutes. An oil-soluble antioxidant (e.g., ascorbyl palmitate or propyl gallate) may also be added to the reacting mixture to prevent auto-oxidation, and is particularly preferred where a non-oxidizing atmosphere is not used.

Specific omega-3 alkyl esters include the ethyl esters of EPA and DHA. For example, the E463808, OMEGA-3/90 (K D Pharma), and Incromega (Croda/Bioriginal) omega-3 ethyl esters are potential omega-3 alkyl esters.

Liquid formulations and methods of the present invention can also be used with fibrates other than fenofibrate, such as clofibrate, bezafibrate, ciprofibrate, beclofibrate, etofibrate, and gemfibrozil.

Liquid formulations of the present invention can, optionally, include non-omega-3 oils. For example, one or more non-omega-3 oils can be used in combination with or in place of one or more omega-3 oils in the vehicle for fenofibrate solubilization.

In some embodiments, a liquid formulation of the present invention may be substantially homogeneous. In some embodiments, a liquid formulation may be homogeneous. In some embodiments, a liquid formulation may be a homogeneous liquid solution.

In another embodiment, an omega-3 oil contains a low percentage of non-omega-3 oil. According to the present invention, an omega-3 oil has a low percentage of non-omega-3 oil when it comprises less than about 25.00, 24.00, 23.00, 22.00, 21.00, 20.00, 19.00, 18.00, 17.00, 16.00, 15.00, 14.00, 13.00, 12.00, 11.00, 10.00, 9.00, 8.00, 7.00, 6.00, 5.00, 4.00, 3.00, 2.00, or 1.00 percent w/w non-omega-3 oil. For example, an omega-3 ethyl ester can comprise about 90 percent omega-3 ethyl esters and about 10 percent non-omega-3 ethyl esters.

Oil purity is an important aspect of the present invention. Oil purity is defined as a percentage (e.g., by volume or by weight) of one component with respect to the entire oil composition. Several examples of oil components include, but are not limited to, monoglycerides, diglycerides, triglycerides, free acids, esters, and derivatives, precursors, and salts thereof. For example, an ester oil with a purity of 95 percent by weight comprises at least 95 percent w/w esters. The remaining percentage may comprise free acids, mono- di- and/or triglycerides, or other components. As another example, an omega-3 ester oil with a purity of 90 percent by weight comprises at least 90 percent omega-3 esters and the remaining percentage can comprise any one or more of other oil components. A mixture of species of one component (e.g., C₈ and C₁₀ esters) need not be discerned in the determination of purity. However, a distinction of specific species within a component (e.g., C₈ and C₁₀ esters) can also be included in specific embodiments of the present invention.

According to the present invention, omega-3 oils with a purity greater than about 85.00 percent, 90.00 percent, 91.00 percent, 92.00 percent, 93.00 percent, 94.00 percent, 95.00 percent, 96.00 percent, 97.00 percent, 98.00 percent, 99.00 percent or more can be used, for example, in a liquid formulation. Omega-3 oils, specifically with a high purity of omega-3 esters, can be used. According to the present invention, omega-3 oils with a high purity comprise greater than about 85.00 percent, 90.00 percent, 91.00 percent, 92.00 percent, 93.00 percent, 94.00 percent, 95.00 percent, 96.00 percent, 97.00 percent, 98.00 percent, 99.00 percent or more of one component by weight or by volume. Omega-3 esters include, but are not limited to, esters of EPA and DHA. Omega-3 esters also include omega-3 ethyl esters.

Oil composition is another important aspect of the present invention. Oil composition can be described as both the species and the components of an oil. Species include specific omega-3 oils such as, but not limited to, EPA, DHA, linoleic acid, linolenic acid, etc. Components include, but are not limited to, monoglycerides, diglycerides, triglycerides, free acids, esters, and derivatives, precursors, and salts thereof. For example, E463808 comprises about 46% EPA and about 38% DHA (mass percent) as ethyl esters. The remaining portion consists essentially of omega-3 oils other than EPA and DHA and other non-omega-3 oils. Other commercially available omega-3 oils contain higher or lower levels of total EPA and DHA as components such as monoglycerides, diglycerides, triglycerides, esters, free acids, etc. or mixtures thereof. For example, omega-3 oils with a composition comprising a mass percent of EPA and DHA equal to or greater than about 55.00 percent, about 75.00 percent, or about 80.00 percent can be used.

Omega-3 oils can be extracted and/or purified from several natural sources, and such processes are described in the art. Omega-3 oils can also be purchased from several commercial vendors, including, but not limited to, Croda International (England), Bioriginal Food and Science Corp. (Canada), Ocean Nutrition Canada (Canada), and Pronova Biocare (Norway).

Mixtures of omega-3 alkyl esters with other components of omega-3 oil (e.g., fatty acids, triglycerides) are not preferred according to the present invention. Fenofibrate solubility is shown, herein, to be maximized in pure omega-3 alkyl esters. Oils containing highly pure or pure alkyl esters are described in the present invention.

In another embodiment, the purity of omega-3 esters or omega-3 alkyl esters is at least about 50.00 percent by weight, at least about 60.00 percent by weight, at least about 70.00 percent by weight, at least about 75.00 percent by weight, at least about 80.00 percent by weight, or at least about 85.00 percent by weight. In another embodiment, the purity of omega-3 esters or omega-3 alkyl esters is about 25.00, 30.00, 35.00, 40.00, 45.00, 50.00, 55.00, 60.00, 65.00, 70.00, 75.00, 80.00, 85.00, 90.00, 95.00, 99.00 percent or more by weight. In another embodiment, the purity of omega-3 esters or omega-3 alkyl esters is between about 25.00 and about 100.00 percent by weight, between about 40.00 and about 100.00 percent by weight, between about 50.00 and about 100.00 percent by weight, between about 60.00 and about 100.00 percent by weight, between about 70.00 and about 100.00 percent by weight, between about 75.00 and about 100.00 percent by weight, between about 75.00 and about 95.00 percent by weight, between about 75.00 and about 90.00 percent by weight, or between about 80.00 and about 85.00 percent by weight. In another embodiment, the purity of omega-3 esters or omega-3 alkyl esters is about 100.00 percent by weight, about 99.00 percent by weight, about 96.00 percent by weight, about 92.00 percent by weight, about 90.00 percent by weight, about 85.00 percent by weight, about 80.00 percent by weight, about 75.00 percent by weight, about 70.00 percent by weight, about 65.00 percent by weight, about 60.00 percent by weight, about 55.00 percent by weight, or about 50.00 percent by weight.

In another embodiment, the oil composition comprising EPA and DHA is at least about 50.00 percent by weight, at least about 60.00 percent by weight, at least about 70.00 percent by weight, at least about 75.00 percent by weight, at least about 80.00 percent by weight, or at least about 84.00 percent by weight. In another embodiment, the oil composition comprising EPA and DHA is about 25.00, 30.00, 35.00, 40.00, 45.00, 50.00, 55.00, 60.00, 65.00, 70.00, 75.00, 80.00, 85.00, 90.00, or 95.00 percent by weight. In another embodiment, the oil composition comprising EPA and DHA is between about 25.00 and about 95.00 percent by weight, between about 40.00 and about 95.00 percent by weight, between about 50.00 and about 95.00 percent by weight, between about 60.00 and about 95.00 percent by weight, between about 70.00 and about 95.00 percent by weight, between about 75.00 and about 95.00 percent by weight, between about 75.00 and about 90.00 percent by weight, between about 75.00 and about 85.00 percent by weight, or between about 80.00 and about 85.00 percent by weight. In another embodiment, the oil composition comprising EPA and DHA is about 99.00 percent by weight, about 96.00 percent by weight, about 92.00 percent by weight, about 90.00 percent by weight, about 84.00 percent by weight, about 80.00 percent by weight, about 75.00 percent by weight, about 70.00 percent by weight, about 65.00 percent by weight, about 60.00 percent by weight, about 55.00 percent by weight, or about 50.00 percent by weight.

In another embodiment, the omega-3 ester or omega-3 alkyl ester has about a 23:19 ratio of EPA:DHA, about a 75:11 ratio of EPA:DHA, about a 95:1 ratio of EPA:DHA, about a 9:2 ratio of EPA:DHA, about a 10:1 ratio of EPA:DHA, about a 5:1 ratio of EPA:DHA, about a 3:1 ratio of EPA:DHA, about a 2:1 ratio of EPA:DHA, about a 1:1 ratio of EPA:DHA, about a 1:2 ratio of EPA:DHA, about a 1:3 ratio of EPA:DHA, or about a 1:5 ratio of EPA:DHA. In another embodiment, the omega-3 ester or omega-3 alkyl ester has about a 95:1 ratio of EPA:DHA, about a 75:1 ratio of EPA:DHA, about a 50:1 ratio of EPA:DHA, about a 25:1 ratio of EPA:DHA, about a 20:1 ratio of EPA:DHA, about a 15:1 ratio of EPA:DHA, about a 10:1 ratio of EPA:DHA, about a 7.5:1 ratio of EPA:DHA, about a 5:1 ratio of EPA:DHA, about a 4:1 ratio of EPA:DHA, about a 3:1 ratio of EPA:DHA, about a 2:1 ratio of EPA:DHA, about a 1.5:1 ratio of EPA:DHA, about a 1:1 ratio of EPA:DHA, about a 1:1.5 ratio of EPA:DHA, about a 1:2 ratio of EPA:DHA, about a 1:3 ratio of EPA:DHA, or about a 1:5 ratio of EPA:DHA. In another embodiment, the omega-3 ester or omega-3 alkyl ester has from about a 95:1 ratio to about a 1:5 ratio of EPA:DHA, from about a 50:1 ratio to about a 1:1 ratio of EPA:DHA, from about a 25:1 ratio to about a 1:1 ratio of EPA:DHA, from about a 10:1 ratio to about a 1:1 ratio of EPA:DHA, from about a 5:1 ratio to about a 1:1 ratio of EPA:DHA, from about a 3:1 ratio to about a 1:1 ratio of EPA:DHA, from about a 2:1 ratio to about a 1:1 ratio of EPA:DHA, or from about a 1.5:1 ratio to about a 1:1 ratio of EPA:DHA. In another embodiment, the omega-3 ester or omega-3 alkyl ester has at least about a 1:5 ratio of EPA:DHA, at least about a 1:1 ratio of EPA:DHA, at least about a 1.5:1 ratio of EPA:DHA, at least about a 2:1 ratio of EPA:DHA, at least about a 3:1 ratio of EPA:DHA, at least about a 5:1 ratio of EPA:DHA, or at least about a 10:1 ratio of EPA:DHA.

In another embodiment, any one or more of the above mentioned or other specific ratios, compositions, or purities of omega-3 oil may be specifically excluded from the present invention. For example, EPA:DHA ratios of 3.3:2, 2.1:1, 3.1:2, 1.9:1, 1.7:1, 1.4:1, 1.1:1, 1:1, and 1:1.8 may be specifically excluded from the present invention. EPA:DHA ratios of from about 1:1 to about 2:1 may also be specifically excluded. In addition, omega-3 oils comprising compositions with, for example, about 80.20, 83.40, 83.70, 86.60, 87.70, or 90.20 percent by weight from EPA and DHA may be specifically excluded from the present invention. An omega-3 oil comprising 90.00 percent (w/w) omega-3 ethyl esters with 46.00 percent EPA and 38.00 percent DHA (e.g., OMACOR®) may be specifically excluded from the present invention. Omega-3 oils comprising an EPA:DHA ratio equal to or greater than 2:1 may be specifically excluded from the present invention. For example, omega-3 oils with an EPA:DHA ratio of about 2:1, 2.5:1, 3:1, 3.5:1, 4:1, 4.5:1, 5:1 or higher may be specifically excluded. Omega-3 oils comprising EPA and DHA in an amount greater than or equal to 75.00, 80.00, 85.00, 90.00, 91.00, 92.00, 93.00, 94.00, or 95.00 percent by weight may be specifically excluded. Omega-3 oils comprising an EPA:DHA ratio equal to about 1:5, 4.5:1, 95:1, 7.5:1, or 1.21:1 may be specifically excluded from the present invention. Other commercially available omega-3 oils may also be specifically excluded according to the present invention including, but not limited to, those available from Croda International (England), Bioriginal Food and Science Corp. (Canada), Ocean Nutrition Canada (Canada), and Pronova Biocare (Norway).

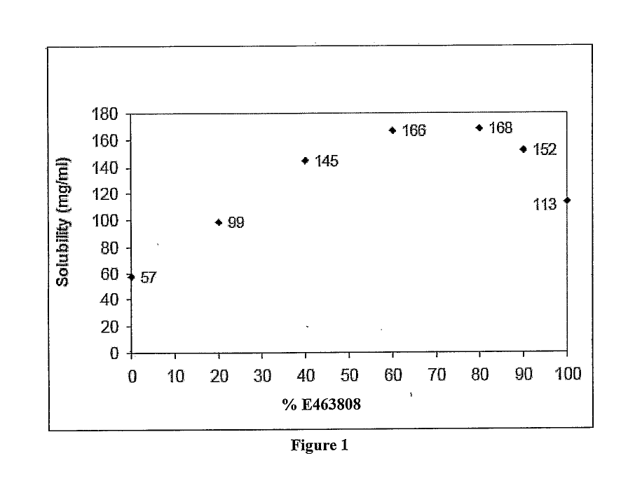

Surprisingly, formulations and medicaments of omega-3 oil and fenofibrate have shown an unexpected increase in fenofibrate solubility when an alcohol is added to the liquid formulation. An alcohol content of about 10.00, 15.00, 20.00, 25.00, 30.00, 35.00, or 40.00 percent by volume is shown to maximize this increased solubilization (See FIG. 1). For example, such an alcohol is ethanol. Another alcohol is glycerol. Alcohols may have one, two, or three or more —OH groups per molecule. One or more alcohols may be excluded from the present invention. In another embodiment, 1,2-propylene glycol is specifically excluded from the present invention. In another embodiment, a single dosage form comprising a liquid formulation of the present invention comprises less than 75 mg 1,2-propylene glycol. In another embodiment, a single dosage form comprising a liquid formulation of the present invention comprises less than 50 mg 1,2-propylene glycol. In another embodiment, a single dosage form comprising a liquid formulation of the present invention comprises less than 25 mg 1,2-propylene glycol. In another embodiment, a single dosage form comprising a liquid formulation of the present invention comprises less than 10 mg 1,2-propylene glycol. In another embodiment, a single dosage form comprising a liquid formulation of the present invention comprises less than 5 mg 1,2-propylene glycol. In another embodiment, a liquid formulation comprises an alcohol which is miscible with the omega-3 oil.

Unless otherwise indicated, reports and discussions herein of fenofibrate solubility in solvents, mixtures, and liquid formulations of the invention are considered to be at 25 degrees C.

In another embodiment, the present invention provides a method for increasing the solubility of fenofibrate in an omega-3 oil, comprising adding an alcohol to said omega-3 oil.

In another embodiment, the fenofibrate solubility in a liquid formulation comprising an omega-3 oil and fenofibrate is increased by at least 10.00 percent by incorporating at least about 5.00, 10.00, 15.00, 20.00, 25.00, 30.00, 35.00, or 40.00 percent by volume of an alcohol. In another embodiment, the fenofibrate solubility in a liquid formulation comprising an omega-3 oil and fenofibrate is increased by at least 20.00 percent by incorporating at least about 5.00, 10.00, 15.00, 20.00, 25.00, 30.00, 35.00, or 40.00 percent by volume of an alcohol. In another embodiment, the fenofibrate solubility in a liquid formulation comprising an omega-3 oil and fenofibrate is increased by at least 30.00 percent by incorporating at least about 5.00, 10.00, 15.00, 20.00, 25.00, 30.00, 35.00, or 40.00 percent by volume of an alcohol. In another embodiment, the fenofibrate solubility in a liquid formulation comprising an omega-3 oil and fenofibrate is increased by at least 40.00 percent by incorporating at least about 5.00, 10.00, 15.00, 20.00, 25.00, 30.00, 35.00, or 40.00 percent by volume of an alcohol. In another embodiment, the fenofibrate solubility in a liquid formulation comprising an omega-3 oil and fenofibrate is increased by at least 50.00 percent by incorporating at least about 5.00, 10.00, 15.00, 20.00, 25.00, 30.00, 35.00, or 40.00 percent by volume of an alcohol. In another embodiment, the fenofibrate solubility in a liquid formulation comprising an omega-3 oil and fenofibrate is increased by at least 60.00 percent by incorporating at least about 5.00, 10.00, 15.00, 20.00, 25.00, 30.00, 35.00, or 40.00 percent by volume of an alcohol.

In another embodiment, a liquid formulation of the present invention comprises at least about 10 percent by weight of an alcohol. For example, a liquid formulation of the present invention comprises at least about 10, 11, 12, 13, 14, 15, 16, 17, 18, 19, or 20 percent by weight of an alcohol.

In another embodiment, a liquid formulation comprises an omega-3 oil, fenofibrate, and an amount of alcohol sufficient to increase the solubility of said fenofibrate by at least about 2.50 percent, 5.00 percent, 10.00 percent, 15.00 percent, 20.00 percent, 25.00 percent, 30.00 percent, 35.00 percent, 40.00 percent, 45.00 percent, 50.00 percent, 55.00 percent, or 60.00 percent over that of the same formulation without alcohol.

It has also been discovered, surprisingly, that a particular form (component) of omega-3 oil is superior in solubilizing fenofibrate. Esters of omega-3 oil have shown greater solubilization power than other forms of omega-3, such as triglycerides. As shown in the exemplification, omega-3 alkyl esters have shown higher solubility of fenofibrate. The employment of both omega-3 alkyl esters and an alcohol in a liquid formulation of the present invention have shown greatly unexpected improvements in fenofibrate solubility. The total amount of EPA and DHA is a factor influencing the solubility of fenofibrate. An increase in the amount of EPA and DHA in a liquid formulation results in an increase in fenofibrate solubility.

In another embodiment, a liquid formulation of the present invention comprises at least about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, 85.00, 86.00, 87.00, 88.00, 89.00, or 90.00% by weight of an omega-3 ester oil and a fenofibrate solubility of about 100, 101, 102, 103, 104, 105, 106, 107, 108, 109, or 110 mg/mL at 25 degrees C.

In another embodiment, a liquid formulation of the present invention comprises at least about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, 85.00, 86.00, 87.00, 88.00, 89.00, or 90.00% by weight of an omega-3 ester oil and a fenofibrate solubility of about 100, 101, 102, 103, 104, 105, 106, 107, 108, 109, 110, 111, 112, 113, 114, 115, 116, 117, 118, 119, or 120 mg/mL at 25 degrees C.

In another embodiment, a liquid formulation of the present invention comprises at least about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, 85.00, 86.00, 87.00, 88.00, 89.00, or 90.00% by weight of an omega-3 ester oil and a fenofibrate solubility of about 110, 111, 112, 113, 114, 115, 116, 117, 118, 119, or 120 mg/mL at 25 degrees C.

In another embodiment, a liquid formulation of the present invention comprises at least about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by weight of an omega-3 ester oil, about 5.00, 6.00, 7.00, 8.00, 9.00, 10.00, 11.00, 12.00, 13.00, 14.00, 15.00, 16.00, 17.00, 18.00, 19.00, or 20.00% by weight C₁ to C₄ alcohol, and a fenofibrate solubility of about 100, 105, 110, 115, 120, 125, 130, 135, 140, 145, 150, 155, 160, 165, or 170 mg/mL at 25 degrees C.

In another embodiment, a liquid formulation of the present invention comprises at least about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, 85.00, 86.00, 87.00, 88.00, 89.00, or 90.00% by weight of an omega-3 ester oil and a fenofibrate solubility of about from 100 to 110 mg/mL at 25 degrees C.

In another embodiment, a liquid formulation of the present invention comprises at least about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, 85.00, 86.00, 87.00, 88.00, 89.00, or 90.00% by weight of an omega-3 ester oil and a fenofibrate solubility of about from 100 to 120 mg/mL at 25 degrees C.

In another embodiment, a liquid formulation of the present invention comprises at least about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, 85.00, 86.00, 87.00, 88.00, 89.00, or 90.00% by weight of an omega-3 ester oil and a fenofibrate solubility of about from 110 to 120 mg/mL at 25 degrees C.

In another embodiment, a liquid formulation of the present invention comprises at least about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by weight of an omega-3 ester oil, about 5.00, 6.00, 7.00, 8.00, 9.00, 10.00, 11.00, 12.00, 13.00, 14.00, 15.00, 16.00, 17.00, 18.00, 19.00, or 20.00% by weight C₁ to C₄ alcohol, and a fenofibrate solubility of about from 100 to 170 mg/mL at 25 degrees C.

In another embodiment, a liquid formulation of the present invention comprises at least about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by weight of an omega-3 ester oil, about 5.00, 6.00, 7.00, 8.00, 9.00, 10.00, 11.00, 12.00, 13.00, 14.00, 15.00, 16.00, 17.00, 18.00, 19.00, or 20.00% by weight C₁ to C₄ alcohol, and a fenofibrate solubility of about from 120 to 170 mg/mL at 25 degrees C.

In another embodiment, a liquid formulation of the present invention comprises at least about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by weight of an omega-3 ester oil, about 5.00, 6.00, 7.00, 8.00, 9.00, 10.00, 11.00, 12.00, 13.00, 14.00, 15.00, 16.00, 17.00, 18.00, 19.00, or 20.00% by weight C₁ to C₄ alcohol, and a fenofibrate solubility of about from 130 to 170 mg/mL at 25 degrees C.

In another embodiment, a liquid formulation of the present invention comprises at least about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by weight of an omega-3 ester oil, about 5.00, 6.00, 7.00, 8.00, 9.00, 10.00, 11.00, 12.00, 13.00, 14.00, 15.00, 16.00, 17.00, 18.00, 19.00, or 20.00% by weight C₁ to C₄ alcohol, and a fenofibrate solubility of at least about 100 mg/mL at 25 degrees C.

In another embodiment, a liquid formulation of the present invention comprises at least about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by weight of an omega-3 ester oil, about 5.00, 6.00, 7.00, 8.00, 9.00, 10.00, 11.00, 12.00, 13.00, 14.00, 15.00, 16.00, 17.00, 18.00, 19.00, or 20.00% by weight C₁ to C₄ alcohol, and a fenofibrate solubility of at least about 110 mg/mL at 25 degrees C.

In another embodiment, a liquid formulation of the present invention comprises at least about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by weight of an omega-3 ester oil, about 5.00, 6.00, 7.00, 8.00, 9.00, 10.00, 11.00, 12.00, 13.00, 14.00, 15.00, 16.00, 17.00, 18.00, 19.00, or 20.00% by weight C₁ to C₄ alcohol, and a fenofibrate solubility of at least about 120 mg/mL at 25 degrees C.

In another embodiment, a liquid formulation of the present invention comprises at least about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by weight of an omega-3 ester oil, about 5.00, 6.00, 7.00, 8.00, 9.00, 10.00, 11.00, 12.00, 13.00, 14.00, 15.00, 16.00, 17.00, 18.00, 19.00, or 20.00% by weight C₁ to C₄ alcohol, and a fenofibrate solubility of at least about 130 mg/mL at 25 degrees C.

In another embodiment, a liquid formulation of the present invention comprises at least about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by weight of an omega-3 ester oil, about 5.00, 6.00, 7.00, 8.00, 9.00, 10.00, 11.00, 12.00, 13.00, 14.00, 15.00, 16.00, 17.00, 18.00, 19.00, or 20.00% by weight C₁ to C₄ alcohol, and a fenofibrate solubility of at least about 140 mg/mL at 25 degrees C.

In another embodiment, a liquid formulation of the present invention comprises at least about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by weight of an omega-3 ester oil, about 5.00, 6.00, 7.00, 8.00, 9.00, 10.00, 11.00, 12.00, 13.00, 14.00, 15.00, 16.00, 17.00, 18.00, 19.00, or 20.00% by weight C₁ to C₄ alcohol, and a fenofibrate solubility of at least about 150 mg/mL at 25 degrees C.

In another embodiment, a liquid formulation of the present invention comprises at least about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by weight of an omega-3 ester oil, about 5.00, 6.00, 7.00, 8.00, 9.00, 10.00, 11.00, 12.00, 13.00, 14.00, 15.00, 16.00, 17.00, 18.00, 19.00, or 20.00% by weight C₁ to C₄ alcohol, and a fenofibrate solubility of at least about 160 mg/mL at 25 degrees C.

In another embodiment, a liquid formulation of the present invention comprises at least about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by weight of an omega-3 ester oil, about 5.00, 6.00, 7.00, 8.00, 9.00, 10.00, 11.00, 12.00, 13.00, 14.00, 15.00, 16.00, 17.00, 18.00, 19.00, or 20.00% by weight C₁ to C₄ alcohol, and a fenofibrate solubility of at least about 170 mg/mL at 25 degrees C.

In another embodiment, a liquid formulation of the present invention comprises at least about 15.00, 16.00, 17.00, 18.00, 19.00, 20.00, 21.00, 22.00, 23.00, 24.00, 25.00, 26.00, 27.00, 28.00, 29.00, 30.00, 31.00, 32.00, 33.00, 34.00, 35.00, 36.00, 37.00, 38.00, 39.00, 40.00, 41.00, 42.00, 43.00, 44.00, 45.00, 46.00, 47.00, 48.00, 49.00, 50.00, 51.00, 52.00, 53.00, 54.00, 55.00, 56.00, 57.00, 58.00, 59.00, 60.00, 61.00, 62.00, 63.00, 64.00, 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, 85.00, 86.00, 87.00, 88.00, 89.00, 90.00, 91.00, 92.00, 93.00, 94.00, or 95.00 percent by weight of an omega-3 ester oil, at least about 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1.00, 2.00, 3.00, 4.00, 5.00, 6.00, 7.00, 8.00, 9.00, 10.00, 11.00, 12.00, 13.00, 14.00, 15.00, 16.00, 17.00, 18.00, 19.00, 20.00, 25.00, or 30.00 percent by weight alcohol, and at least about 1.00, 2.00, 3.00, 4.00, 5.00, 6.00, 7.00, 8.00, 9.00, 10.00, 11.00, 12.00, 13.00, 14.00, 15.00, 16.00, 17.00, 18.00, 19.00, or 20.00 percent by weight of fenofibrate.

In another embodiment, a liquid formulation of the present invention comprises at least about 15.00, 16.00, 17.00, 18.00, 19.00, 20.00, 21.00, 22.00, 23.00, 24.00, 25.00, 26.00, 27.00, 28.00, 29.00, 30.00, 31.00, 32.00, 33.00, 34.00, 35.00, 36.00, 37.00, 38.00, 39.00, 40.00, 41.00, 42.00, 43.00, 44.00, 45.00, 46.00, 47.00, 48.00, 49.00, 50.00, 51.00, 52.00, 53.00, 54.00, 55.00, 56.00, 57.00, 58.00, 59.00, 60.00, 61.00, 62.00, 63.00, 64.00, 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, 85.00, 86.00, 87.00, 88.00, 89.00, 90.00, 91.00, 92.00, 93.00, 94.00, or 95.00 percent by weight of an omega-3 ester oil, less than about 30.00, 25.00, 20.00, 15.00, 10.00, 5.00, or 2.50 percent by weight alcohol, and at least about 1.00, 2.00, 3.00, 4.00, 5.00, 6.00, 7.00, 8.00, 9.00, 10.00, 11.00, 12.00, 13.00, 14.00, 15.00, 16.00, 17.00, 18.00, 19.00, or 20.00 percent by weight of fenofibrate.

In another embodiment, a medium-chain triglyceride such as a caprylic/capric triglyceride (e.g., Neobee® M5 Stepan Company) or a medium chain mono-diglyceride such as caprylic/capric mono-diglyceride (e.g., Capmul® MCM, Abitec Corporation) may be included in a formulation of the invention to facilitate digestion of the formulation or reduce the food effect. In another embodiment, a surfactant may be included in a formulation of the invention to facilitate digestion of the formulation or reduce the food effect.

A surfactant-containing liquid formulation or medicament of the invention comprises a mixture of fenofibrate dissolved in a vehicle comprising an omega-3 ester or omega-3 alkyl ester and, optionally, a C₁ to C₄ alcohol, wherein:

(a) the formulation comprises (i) about 5.00, 6.00, 7.00, 8.00, 9.00, 10.00, 11.00, 12.00, 13.00, 14.00, 15.00, 16.00, 17.00, 18.00, 19.00, or 20.00% by weight of fenofibrate (ii) about 55.00, 56.00, 57.00, 58.00, 59.00, 60.00, 61.00, 62.00, 63.00, 64.00, 65.00, 66.00, 67.00, 68.00, 69.00, 70.00, 71.00, 72.00, 73.00, 74.00, 75.00, 76.00, 77.00, 78.00, 79.00, or 80.00% by weight of an omega-3 ester or omega-3 alkyl ester (iii) about 5.00, 6.00, 7.00, 8.00, 9.00, 10.00, 11.00, 12.00, 13.00, 14.00, 15.00, 16.00, 17.00, 18.00, 19.00, 20.00, 21.00, 22.00, 23.00, 24.00, or 25.00% by weight of a surfactant, and, optionally, (iv) about 5.00, 6.00, 7.00, 8.00, 9.00, 10.00, 11.00, 12.00, 13.00, 14.00, 15.00, 16.00, 17.00, 18.00, 19.00, or 20.00% by weight of a C₁ to C₄ alcohol; and
(b) the solubility of the fenofibrate in the vehicle is about 50, 60, 70, 80, 90, 100, 110, 120, 130, 140, 150, 160, 170, 180, 190, 200, 210, 220, 230, 240, 250, 260, 270, 280, 290, or 300 milligrams per milliliter at 25 degrees C.

In another embodiment, the surfactant-containing liquid formulation or medicament of the invention comprises a C₁ to C₄ alcohol, such as ethanol.

In an alternative embodiment, the surfactant increases the bioavailability of the non-aqueous formulation in the fasted state when compared with the non-aqueous formulation without surfactant.

A surfactant-containing liquid formulation or medicament of the invention comprises a mixture of fenofibrate dissolved in a vehicle comprising an omega-3 ester or omega-3 alkyl ester and, optionally, a C₁ to C₄ alcohol, wherein:

(a) the formulation comprises (i) about 5.00, 6.00, 7.00, 8.00, 9.00, 10.00, 11.00, 12.00, 13.00, 14.00, 15.00, 16.00, 17.00, 18.00, 19.00, or 20.00% by weight of fenofibrate (ii) about 55.00, 56.00, 57.00, 58.00, 59.00, 60.00, 61.00, 62.00, 63.00, 64.00, 65.00, 66.00, 67.00, 68.00, 69.00, 70.00, 71.00, 72.00, 73.00, 74.00, 75.00, 76.00, 77.00, 78.00, 79.00, or 80.00% by weight of an omega-3 ester or omega-3 alkyl ester (iii) about 5.00, 6.00, 7.00, 8.00, 9.00, 10.00, 11.00, 12.00, 13.00, 14.00, 15.00, 16.00, 17.00, 18.00, 19.00, 20.00, 21.00, 22.00, 23.00, 24.00, or 25.00% by weight of a surfactant, and, optionally, (iv) about 5.00, 6.00, 7.00, 8.00, 9.00, 10.00, 11.00, 12.00, 13.00, 14.00, 15.00, 16.00, 17.00, 18.00, 19.00, or 20.00% by weight of a C₁ to C₄ alcohol.

In another embodiment, the surfactant-containing liquid formulation or medicament of the invention comprises a C₁ to C₄ alcohol, such as ethanol.

In another embodiment, the surfactant increases the solubility of the fenofibrate in the non-diluted liquid formulation.

In another embodiment, the surfactant increases the bioavailability of the non-aqueous formulation in the fasted state when compared with the non-aqueous formulation without surfactant, and the surfactant increases the solubility of the fenofibrate in the non-diluted liquid formulation.

In one embodiment of the invention, the surfactant is contained within the solid material of the capsule (i.e., within the gelatin casing or shell of a gelcap). In such an embodiment, the surfactant is prohibited from interacting with the omega-3 oil, the fenofibrate, and any other contents until after the solid capsule structure begins to dissolve (i.e., in vivo or in an aqueous environment).

In another embodiment, a liquid formulation according to the present invention comprises a surfactant with a weight percent less than about 50.00 percent of the total formulation. In another embodiment, a liquid formulation according to the present invention comprises a surfactant with a weight percent less than about 40.00 percent of the total formulation. In another embodiment, a liquid formulation according to the present invention comprises a surfactant with a weight percent less than about 30.00 percent of the total formulation. In another embodiment, a liquid formulation according to the present invention comprises a surfactant with a weight percent less than about 25.00 percent of the total formulation. In another embodiment, a liquid formulation according to the present invention comprises a surfactant with a weight percent less than about 20.00 percent of the total formulation. In another embodiment, a liquid formulation according to the present invention comprises a surfactant with a weight percent less than about 15.00 percent of the total formulation. In another embodiment, a liquid formulation according to the present invention comprises a surfactant with a weight percent less than about 10.00 percent of the total formulation. In another embodiment, a liquid formulation according to the present invention comprises a surfactant with a weight percent less than about 5.00 percent of the total formulation.

A formulation containing a high concentration of surfactant, according to the present invention, is one which has at least 30.00, 35.00, 40.00, 45.00, or 50.00 percent by weight of one or more surfactants. In another embodiment, a liquid formulation according to the present invention comprising a surfactant with a weight percent of about 25.00 or less, has a solubility of fenofibrate equal to or greater than that of formulations containing high concentrations of surfactant. In another embodiment, a liquid formulation according to the present invention comprising a surfactant with a weight percent of about 20.00 or less, has a solubility of fenofibrate equal to or greater than that of formulations containing high concentrations of surfactant. In another embodiment, a liquid formulation according to the present invention comprising a surfactant with a weight percent of about 15.00 or less, has a solubility of fenofibrate equal to or greater than that of formulations containing high concentrations of surfactant. In another embodiment, a liquid formulation according to the present invention comprising a surfactant with a weight percent of about 10.00 or less, has a solubility of fenofibrate equal to or greater than that of formulations containing high concentrations of surfactant. In another embodiment, a liquid formulation according to the present invention comprising a surfactant with a weight percent of about 5.00 or less, has a solubility of fenofibrate equal to or greater than that of formulations containing high concentrations of surfactant.

AUC is the area under the plot of plasma concentration of drug (not logarithm of the concentration) against time after drug administration. The area is conveniently determined by the “trapezoidal rule”: the data points are connected by straight line segments, perpendiculars are erected from the abscissa to each data point, and the sum of the areas of the triangles and trapezoids so constructed is computed. When the last measured concentration (Cₙ, at time tₙ) is not zero, the AUC from tₙ to infinite time is estimated by Cₙ/kₑₗ.

The AUC is of particular use in estimating bioavailability of drugs, and in estimating total clearance of drugs (Cl_{T}). Following single intravenous doses, AUC=D/Cl_{T}, where D is the dose, for single compartment systems obeying first-order elimination kinetics; alternatively, AUC=C₀/kₑₗ, where kₑₗ is the drug elimination rate constant. With routes other than the intravenous, AUC=F·D/Cl_{T}, where F is the absolute bioavailability of the drug.

The AUC of fenofibrate can be used as an indicator of the relative bioavailability of a liquid formulation of the present invention with respect to a reference composition (e.g., TRICOR®).

In another embodiment, the bioavailability of a liquid formulation of the invention is at least as high as that of the 160 mg dose of Tricor®. In one embodiment, a liquid formulation of the present invention which has about a 160 mg dose of fenofibrate per capsule has a bioavailability approximately equal to or higher than that of the 160 mg dose of Tricor®. In another embodiment, a liquid formulation of the present invention which has about a 150 mg dose of fenofibrate per capsule has a bioavailability approximately equal to that of the 160 mg dose of Tricor®. In another embodiment, a liquid formulation of the present invention which has about a 145 mg dose of fenofibrate per capsule has a bioavailability approximately equal to that of the 160 mg dose of Tricor®. In another embodiment, a liquid formulation of the present invention which has about a 140 mg dose of fenofibrate per capsule has a bioavailability approximately equal to that of the 160 mg dose of Tricor®. In another embodiment, a liquid formulation of the present invention which has about a 130 mg dose of fenofibrate per capsule has a bioavailability approximately equal to that of the 160 mg dose of Tricor®. In another embodiment, a liquid formulation of the present invention which has about a 120 mg dose of fenofibrate per capsule has a bioavailability approximately equal to that of the 160 mg dose of Tricor®.

A particular formulation of the invention comprises fenofibrate dissolved in a vehicle at a concentration of about 50, 60, 70, 80, 90, 100, 110, 120, 130, 140, 150, 160, 170, 180, 190, or 200 milligrams of fenofibrate per milliliter of formulation, wherein the vehicle consists of EPA and/or DHA ethyl esters, about 5.00, 6.00, 7.00, 8.00, 9.00, 10.00, 11.00, 12.00, 13.00, 14.00, or 15.00% by volume of ethanol, and a medium-chain triglyceride, and wherein the formulation composition on a weight percentage basis is as follows: about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by weight of EPA and/or DHA ethyl esters, about 5.00, 6.00, 7.00, 8.00, 9.00, 10.00, 11.00, 12.00, 13.00, 14.00, or 15.00% by weight of ethanol, about 5.00, 6.00, 7.00, 8.00, 9.00, 10.00, 11.00, 12.00, 13.00, 14.00, or 15.00% by weight of the medium chain triglyceride, and about 5.00, 6.00, 7.00, 8.00, 9.00, 10.00, 11.00, 12.00, 13.00, 14.00, 15.00, 16.00, 17.00, 18.00, 19.00, or 20.00% by weight of fenofibrate.

Another formulation of the invention comprises fenofibrate dissolved in a vehicle at a concentration of about 50, 60, 70, 80, 90, 100, 110, 120, 130, 140, 150, 160, 170, 180, 190, or 200 milligrams of fenofibrate per milliliter of formulation, wherein the vehicle comprises about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by volume of omega-3 ethyl esters, and about 5.00, 6.00, 7.00, 8.00, 9.00, 10.00, 11.00, 12.00, 13.00, 14.00, 15.00, 16.00, 17.00, 18.00, 19.00, or 20.00% by volume of ethanol, and wherein: (1) the formulation composition on a weight percentage basis is as follows: about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by weight of omega-3 ethyl esters, about 5.00, 6.00, 7.00, 8.00, 9.00, 10.00, 11.00, 12.00, 13.00, 14.00, or 15.00% by weight of ethanol, and about 10.00, 11.00, 12.00, 13.00, 14.00, 15.00, 16.00, 17.00, 18.00, 19.00, or 20.00% by weight of fenofibrate, and (2) the molar ratio of unsaturated moieties contained with the omega-3 ethyl esters to the total moles of omega-3 ethyl ester is about 5 to about 6.

A particular capsule dosage form of the invention comprises fenofibrate relatively uniformly dispersed in a vehicle at a concentration of about 50, 60, 70, 80, 90, 100, 110, 120, 130, 140, 150, 160, 170, 180, 190, or 200 milligrams fenofibrate per milliliter of formulation, wherein the vehicle comprises about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by volume an omega-3 ethyl ester, and about 5.00, 6.00, 7.00, 8.00, 9.00, 10.00, 11.00, 12.00, 13.00, 14.00, 15.00, 16.00, 17.00, 18.00, 19.00, or 20.00% by volume of ethanol, and wherein: (1) the formulation composition on a weight percentage basis is as follows: about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by weight of omega-3 ethyl ester, about 5.00, 6.00, 7.00, 8.00, 9.00, 10.00, 11.00, 12.00, 13.00, 14.00, or 15.00% by weight of ethanol, and about 5.00, 6.00, 7.00, 8.00, 9.00, 10.00, 11.00, 12.00, 13.00, 14.00, 15.00, 16.00, 17.00, 18.00, 19.00, or 20.00% by weight of fenofibrate, and (2) the molar ratio of unsaturated moieties contained with the omega-3 ethyl ester to the total moles of omega-3 ethyl ester is about 5 to about 6.

Another capsule dosage form of the invention comprises fenofibrate relatively uniformly dispersed in a vehicle at a concentration of about 50, 60, 70, 80, 90, 100, 110, 120, 130, 140, 150, 160, 170, 180, 190, or 200 milligrams fenofibrate per milliliter of formulation, wherein the vehicle comprises about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by volume EPA and/or DHA ethyl ester, and about 5.00, 6.00, 7.00, 8.00, 9.00, 10.00, 11.00, 12.00, 13.00, 14.00, 15.00, 16.00, 17.00, 18.00, 19.00, or 20.00% by volume of ethanol, and wherein the formulation composition on a weight percentage basis is as follows: about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by weight of EPA and/or DHA ethyl ester, about 5.00, 6.00, 7.00, 8.00, 9.00, or 10.00% by weight of ethanol, and about 10, 11.00, 12.00, 13.00, 14.00, 15.00, 16.00, 17.00, 18.00, 19.00, or 20.00% by weight of fenofibrate.

In another embodiment, a liquid formulation or medicament of the present invention comprising about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by weight of omega-3 ethyl ester, about 5.00, 6.00, 7.00, 8.00, 9.00, 10.00, 11.00, 12.00, 13.00, 14.00, 15.00, 16.00, 17.00, 18.00, 19.00, or 20.00% by weight of ethanol, and about 5.00, 6.00, 7.00, 8.00, 9.00, 10.00, 11.00, 12.00, 13.00, 14.00, 15.00, 16.00, 17.00, 18.00, 19.00, or 20.00% by weight of fenofibrate completely solubilizes the fenofibrate at 25 degrees C.

In another embodiment, a liquid formulation or medicament of the present invention comprising about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by weight of omega-3 ethyl ester, about 5.00, 6.00, 7.00, 8.00, 9.00, 10.00, 11.00, 12.00, 13.00, 14.00, 15.00, 16.00, 17.00, 18.00, 19.00, or 20.00% by weight of ethanol, and about 5.00, 6.00, 7.00, 8.00, 9.00, 10.00, 11.00, 12.00, 13.00, 14.00, 15.00, 16.00, 17.00, 18.00, 19.00, or 20.00% by weight of fenofibrate has a fenofibrate solubility greater than or equal to about 70 mg/mL at about 4 degrees C. In another embodiment, a liquid formulation of the present invention comprising about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by weight of omega-3 ethyl ester, about 5.00, 6.00, 7.00, 8.00, 9.00, 10.00, 11.00, 12.00, 13.00, 14.00, 15.00, 16.00, 17.00, 18.00, 19.00, or 20.00% by weight of ethanol, and about 5.00, 6.00, 7.00, 8.00, 9.00, 10.00, 11.00, 12.00, 13.00, 14.00, 15.00, 16.00, 17.00, 18.00, 19.00, or 20.00% by weight of fenofibrate has a fenofibrate solubility greater than or equal to about 100 mg/mL at about 10 degrees C. In another embodiment, a liquid formulation of the present invention comprising about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by weight of omega-3 ethyl ester, about 5.00, 6.00, 7.00, 8.00, 9.00, 10.00, 11.00, 12.00, 13.00, 14.00, 15.00, 16.00, 17.00, 18.00, 19.00, or 20.00% by weight of ethanol, and about 5.00, 6.00, 7.00, 8.00, 9.00, 10.00, 11.00, 12.00, 13.00, 14.00, 15.00, 16.00, 17.00, 18.00, 19.00, or 20.00% by weight of fenofibrate has a fenofibrate solubility greater than or equal to about 150 mg/mL at about 22 degrees C. In another embodiment, a liquid formulation of the present invention comprising about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by weight of omega-3 ethyl ester, about 5.00, 6.00, 7.00, 8.00, 9.00, 10.00, 11.00, 12.00, 13.00, 14.00, 15.00, 16.00, 17.00, 18.00, 19.00, or 20.00% by weight of ethanol, and about 5.00, 6.00, 7.00, 8.00, 9.00, 10.00, 11.00, 12.00, 13.00, 14.00, 15.00, 16.00, 17.00, 18.00, 19.00, or 20.00% by weight of fenofibrate has a fenofibrate solubility greater than or equal to about 160 mg/mL at about 25 degrees C. In another embodiment, a liquid formulation of the present invention comprising about 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00% by weight of omega-3 ethyl ester, about 5.00, 6.00, 7.00, 8.00, 9.00, 10.00, 11.00, 12.00, 13.00, 14.00, 15.00, 16.00, 17.00, 18.00, 19.00, or 20.00% by weight of ethanol, and about 5.00, 6.00, 7.00, 8.00, 9.00, 10.00, 11.00, 12.00, 13.00, 14.00, 15.00, 16.00, 17.00, 18.00, 19.00, or 20.00% by weight of fenofibrate has a fenofibrate solubility greater than or equal to about 220 mg/mL at about 33 degrees C.

In another embodiment, a liquid formulation or medicament of the present invention comprising about 80.00, 81.00, 82.00, 83.00, 84.00, 85.00, 86.00, 87.00, 88.00, 89.00, 90.00, 91.00, 92.00, 93.00, 94.00, or 95.00% by weight of omega-3 ethyl ester and about 5.00, 6.00, 7.00, 8.00, 9.00, 10.00, 11.00, 12.00, 13.00, 14.00, 15.00, 16.00, 17.00, 18.00, 19.00, or 20.00% by weight of fenofibrate has a fenofibrate solubility greater than or equal to about 50 mg/mL at about 4 degrees C. In another embodiment, a liquid formulation of the present invention comprising about 80.00, 81.00, 82.00, 83.00, 84.00, 85.00, 86.00, 87.00, 88.00, 89.00, 90.00, 91.00, 92.00, 93.00, 94.00, or 95.00% by weight of omega-3 ethyl ester and about 5.00, 6.00, 7.00, 8.00, 9.00, 10.00, 11.00, 12.00, 13.00, 14.00, 15.00, 16.00, 17.00, 18.00, 19.00, or 20.00% by weight of fenofibrate has a fenofibrate solubility greater than or equal to about 100 mg/mL at about 22 degrees C. In another embodiment, a liquid formulation of the present invention comprising about 80.00, 81.00, 82.00, 83.00, 84.00, 85.00, 86.00, 87.00, 88.00, 89.00, 90.00, 91.00, 92.00, 93.00, 94.00, or 95.00% by weight of omega-3 ethyl ester and about 5.00, 6.00, 7.00, 8.00, 9.00, 10.00, 11.00, 12.00, 13.00, 14.00, 15.00, 16.00, 17.00, 18.00, 19.00, or 20.00% by weight of fenofibrate has a fenofibrate solubility greater than or equal to about 150 mg/mL at about 33 degrees C.

In another embodiment, a method of increasing the solubility of fenofibrate in a liquid formulation or a medicament containing an omega-3 oil is provided by adding from about 1 to about 25 percent by volume of an ester-based oil. In one specific embodiment, the omega-3 oil exists as triglycerides. In another specific embodiment, the omega-3 oil exists as mono-diglycerides. In another specific embodiment, the omega-3 oil exists as free acids. In another specific embodiment, the omega-3 oil exists as phospholipids. In another specific embodiment, the omega-3 oil exists as a mixture of triglycerides, mono-diglycerides, and free acids. In another specific embodiment, the omega-3 oil exists as a mixture of triglycerides and mono-diglycerides. In another specific embodiment, the omega-3 oil exists as a mixture of triglycerides and free acids. In another specific embodiment, the omega-3 oil exists as a mixture of mono-diglycerides and free acids.

In another embodiment, a liquid formulation or medicament of the present invention can be stored for up to 8 weeks at about 25 degrees C. with no detectable degradation of fenofibrate. In another embodiment, a liquid formulation of the present invention can be stored for up to 12 weeks at about 25 degrees C. with no detectable degradation of fenofibrate. In another embodiment, a liquid formulation of the present invention can be stored for up to 16 weeks at about 25 degrees C. with no detectable degradation of fenofibrate.

In some formulations, it is possible for the fenofibrate, or a portion thereof, to precipitate out of solution during storage. This can be caused by, for example, a storage temperature significantly below room temperature. In another embodiment, a liquid formulation of the present invention further comprises pharmaceutically acceptable precipitation nuclei to promote the crystallization of multiple, small crystals. In another embodiment, a liquid formulation of the present invention is administered in slow-dissolving gelatin capsules, so as to increase the duration of time such capsules remain intact in the patient's stomach. For example, a slow-dissolving gelatin capsule can take 5, 6, 7, 8, 9, 10, 11, 12, 13, 14, 15, 16, 17, 18, 19, or 20 minutes or more to open in vivo. In another embodiment, a liquid formulation comprises pharmaceutically acceptable precipitation nuclei and is administered in slow-dissolving gelatin capsules. In another embodiment, a liquid formulation comprises pharmaceutically acceptable precipitation nuclei and is administered in slow-dissolving gelatin capsules so as to effectively provide completely solubilized fenofibrate upon capsule dissolution in vivo. In another embodiment, a liquid formulation of the present invention maintains fenofibrate in solution at a temperature of about 22 degrees C. In another embodiment, a liquid formulation of the present invention maintains fenofibrate in solution at a temperature of about 18 degrees C. In another embodiment, a liquid formulation of the present invention maintains fenofibrate in solution at a temperature of about 15 degrees C. In another embodiment, a liquid formulation of the present invention maintains fenofibrate in solution at a temperature of about 12 degrees C. In another embodiment, a liquid formulation of the present invention maintains fenofibrate in solution at a temperature of about 15 degrees C. and a fenofibrate concentration of at least 100 mg/mL. In another embodiment, a liquid formulation of the present invention maintains fenofibrate in solution at a temperature of about 15 degrees C. and a fenofibrate concentration of at least 110 mg/mL. In another embodiment, a liquid formulation of the present invention maintains fenofibrate in solution at a temperature of about 15 degrees C. and a fenofibrate concentration of at least 120 mg/mL. In another embodiment, a liquid formulation of the present invention maintains fenofibrate in solution at a temperature of about 15 degrees C. and a fenofibrate concentration of at least 130 mg/mL. In another embodiment, a liquid formulation of the present invention maintains fenofibrate in solution at a temperature of about 15 degrees C. and a fenofibrate concentration of at least 140 mg/mL. In another embodiment, a liquid formulation of the present invention maintains fenofibrate in solution at a temperature of about 15 degrees C. and a fenofibrate concentration of at least 150 mg/mL. In another embodiment, a liquid formulation of the present invention maintains fenofibrate in solution from its initial manufacture, through storage and handling, to administration.

In another embodiment, a method of treating a patient suffering from hyperlipidemia is provided. This method comprises administering to the patient a therapeutically effective amount of a liquid formulation of the present invention. In another embodiment, the patient is a human.

The liquid formulations of the present invention can be prepared according to any one or more methods available in the art. For example, in one embodiment comprising omega-3 oil, fenofibrate, ethanol, and one or more surfactants, appropriate amounts of said formulation components can be mixed together at room temperature or at a slightly elevated temperature. Where one or more formulation components contain a solid which has precipitated from solution (e.g., a surfactant), such a component can be heated and mixed so as to induce resolubilization prior to combining with the remaining formulation components.

A therapeutically acceptable daily dosage of omega-3 oil has been recommended or considered via several national and international groups including, but not limited to, the American Heart Association (AHA) and the International Society for the Study of Fatty Acids and Lipids (ISSFAL). Table 1 includes daily dosage amounts of omega-3 as considered/recommended via several organizations.

TABLE 1
Daily dosages of omega-3

| Omega-3 dose (grams)/day | Comment |
|---|---|
| 0.65 | ISSFAL consideration (1999) |
| 1.0 | AHA recommended (2000, 2004) |
| 1.8 | Omacor ® dose |
| 3.0 | FDA limit on daily consumption, |
| | general population |
| 3.6 | Omacor ® dose |

In another embodiment, the present invention provides a novel polymorph of fenofibrate.

In another embodiment, the present invention provides a method of making a polymorph of fenofibrate, comprising:
(a) combining fenofibrate with one or more components so as to form a solution of fenofibrate;
(b) decreasing the temperature of said solution; and
(c) collecting a precipitated solid.

Liquid formulations of the invention may comprise any one polymorph of fenofibrate or a mixture of two or more polymorphs of fenofibrate. For example, a liquid formulation of the present invention may be prepared from fenofibrate (Form I), fenofibrate (Form II), or a mixture of Forms I and II.

Typical dosage forms of the invention comprise from about 10 mg to about 1000 mg, or an amount of from about 25 mg to about 500 mg, or an amount of from 40 mg to 400 mg, or an amount of from about 50 mg to about 200 mg of fenofibrate. For example, dosage forms comprising 30, 40, 50, 60, 70, 80, 90, 100, 110, 120, 130, 140, 145, 150, 160, 170, 180, 190, or 200 mg fenofibrate are included. More specifically, doses include 50, 100, 145, 150, and 160 mg of fenofibrate.

Liquid formulations of the present invention, optionally, can be administered in soft gelatin capsules. Such soft gelatin capsules can be in any shape, for example, oval or oblongs. The volume of such capsules can be between about 0.5 mL and about 1.5 mL. For example, about 0.50, 0.55, 0.60, 0.65, 0.70, 0.75, 0.80, 0.85, 0.90, 0.95, 1.00, 1.05, 1.10, 1.15, 1.20, 1.25, 1.30, 1.35, 1.40, 1.45, or 1.50 mL. In one embodiment, one dose consists of a single capsule. In another embodiment, one dose consists of two capsules. In another embodiment, one dose consists of three or more capsules. Optionally, each dose can be packaged individually in a blister-pack. In another embodiment, the soft gelatin material is both chemically and physically stable while in contact with a liquid formulation of the invention. In another embodiment, the soft gelatin material prevents the alcohol in the liquid formulation from escaping the capsule. In another embodiment, the soft gelatin material prevents a significant amount of the alcohol in the liquid formulation from escaping the capsule.

All aforementioned ranges (e.g., 65.00, 66.00, 67.00, 68.00, 69.00, 70.00, 71.00, 72.00, 73.00, 74.00, 75.00, 76.00, 77.00, 78.00, 79.00, 80.00, 81.00, 82.00, 83.00, 84.00, or 85.00) of percent identity are to be taken as including, and providing written description and support for, any fractional percentage, in intervals of 0.01%.

It is generally practiced that the process for preparing the formulations include the use of a purge of an inert gas. Such inert gases are for example, nitrogen, argon, and the like. The use of an isolator to maintain low oxygen conditions is desirable, but not required for storage of the present formulation.

These and other embodiments of the invention are illustrated further in the following examples, which are illustrative and in no way limiting.

### EXEMPLIFICATION

### Materials and Methods

### Powder X-Ray Diffraction

All X-ray powder diffraction patters were obtained using a D/Max Rapid X-ray Diffractometer (Rigaku/MSC, The Woodlands, Tex., U.S.A.) equipped with a copper source (Cu/K_{α}1.5406 A), manual x-y stage, and 0.3 mm collimator. A sample was loaded into a 0.3 mm quartz capillary tube (Charles Supper Company, Natick, Mass., U.S.A.) by sectioning off the closed end of the tube and tapping the small, open end of the capillary tube into a bed of the powdered sample or into the sediment of a slurried sample. The precipitate can be amorphous or crystalline. The loaded capillary tube was mounted in a holder that was placed and fitted into the x-y stage. A diffractogram was acquired using control software (RINT Rapid Control Software, Rigaku Rapid/XRD, version 1.0.0 (©1999 Rigaku Co.)) under ambient conditions at a power setting of 46 kV at 40 mA in transmission mode, while oscillating about the omega-axis from 0-5 degrees at 1 degree/second, and spinning about the phi-axis over 360 degrees at 2 degrees/second. The exposure time was 15 minutes unless otherwise specified.

The diffractogram obtained was integrated of 2-theta from 2-60 degrees and chi (1 segment) from 0-36 degrees at a step size of 0.02 degrees using the cyllnt utility in the RINT Rapid display software (RINT Rapid display software, version 1.18 (Rigaku/MSC)) provided by Rigaku with the instrument. The dark counts value was set to 8 as per the system calibration by Rigaku. No normalization or omega, chi or phi offsets were used for the integration.

The relative intensity of peaks in a diffractogram were determined by visual comparison of the peaks in the diffractogram.

Unless otherwise specified, the term fenofibrate refers to fenofibrate Form I in the Exemplification.

### Solubility Measurements Via Ultraviolet (UV) Absorption

First, a calibration curve was constructed by preparing known concentrations of fenofibrate in absolute ethanol in volumetric flasks. At each concentration, 200 microliters of the solution was transferred into a 96-well clear bottom UV plate. The sample absorbance was measured at 280 nm (unless otherwise noted) in a UV spectrophotometer. It was found that the absorbance vs. concentration correlation was linear to at least 100 micrograms/mL.

To measure the fenofibrate concentration in the sample, a small aliquot was taken and diluted (typically 2000-fold) with absolute ethanol in a volumetric flask to a final approximate concentration of less than 100 micrograms/mL. The absorbance at 280 nm (unless otherwise noted) is measured and the solubility is calculated based on the calibration curve.

### Example 1

### Solubility of Fenofibrate in Different Liquid Vehicles

Saturated solutions of fenofibrate in various liquid vehicles were prepared in 1.5 mL glass vials by stepwise addition of fenofibrate powder to approximately 0.5-1 mL of liquid vehicle. If the powder dissolved completely, more fenofibrate was added until an excess of powder was observed. The samples were then stirred overnight at 25° C. controlled temperature before being filtered through a 0.2 micrometer PVDF syringe filter. The filtrate was diluted with n-heptane and analyzed via normal phase HPLC.

Table 2 summarizes the solubility of Fenofibrate in various liquid vehicles.

TABLE 2
Solubility of fenofibrate in various liquid vehicles

| | | Solubility (mg/ml, |
|---|---|---|
| No. | Mixture | at 25 degrees C.) |
| 1 | 100% E9501EE** | 107 |
| 2 | 100% E463808* | 113 |
| 3 | 90:10 E463808:Ethanol* | 152 |
| 4 | 80:20 E463808:Ethanol* | 168 |
| 5 | 60:40 E463808:Ethanol* | 166 |
| 6 | 40:60 E463808:Ethanol* | 145 |
| 7 | 20:80 E463808:Ethanol* | 99 |
| 8 | 100% Ethanol | 57 |
| 9 | 80:10:10 E463808:Ethanol:Labrafac CC* | 148 |
| 10 | 100% Omegabrite | 113 |
| 11 | 100% Myvacet 9-45 | 115 |
| 12 | 100% Epax 1050TG | 76 |
| 13 | 100% Epax 4510TG | 80 |
| 14 | 100% Cod liver oil | 52 |
| 15 | 100% Natural fish oil | 55 |
| 16 | 100% Flaxseed oil | 57 |
| 17 | 100% Flax-borage | 59 |

*E463808 comprises 46 percent EPA, 38 percent DHA, and 8 percent other omega-3's as ethyl esters (mass percent)
**E9501EE comprises 95 percent EPA, 1 percent DHA, as ethyl esters (mass percent)

Based on available composition data, Table 3 below compares fenofibrate solubility and omega-3 content in different vehicles

TABLE 3
Fenofibrate solubility and omega-3 content (mass percent) in various vehicles

| | | | | | Solubility (mg/mL) |
|---|---|---|---|---|---|
| | EPA % | DHA % | Other omega-3% | Total % | at 25 deg C. |
| Cod Liver Oil | 11 | 11 | 0 | 22 | 52 |
| Natural Fish Oil | 18 | 12 | 0 | 30 | 55 |
| Flax Seed Oil | 0 | 0 | 50 | 50 | 57 |
| Flax-Borage Oil | 7 | 5 | 50 | 62 | 59 |
| EPAX ® 1050 TG | 10 | 50 | 0 | 60 | 76 |
| EPAX ® 4510 TG | 45 | 10 | 0 | 55 | 80 |
| E9501EE** | 95 | 1 | 0 | 96 | 107 |
| Omegabrite ® | 75 | 11 | 6 | 92 | 113 |
| E463808* | 46 | 38 | 8 | 92 | 113 |

*E463808 comprises 46 percent EPA, 38 percent DHA, and 8 percent other omega-3's (mass percent) as ethyl esters
**E9501EE comprises 95 percent EPA, 1 percent DHA (mass percent) as ethyl esters

It is believed that, among other factors, fenofibrate solubility in omega-3 oils may also be proportional to the number of double-bonds present in the vehicle. Using available composition data from E463808 and E9501EE omega-3 oils, Table 4 below shows the estimated moles of double bonds per gram of vehicle and their corresponding fenofibrate solubility:

TABLE 4
Estimated double bonds per gram of vehicle

| Vehicle | Moles double bond/g | Fenofibrate solubility (mg/ml) |
|---|---|---|
| E463808 | 0.01514 | 113 |
| E9501EE | 0.01506 | 107 |

FIG. 1 shows the solubility of fenofibrate in E463808/Ethanol mixtures at 25 degrees C. FIG. 1 illustrates the effect of ethanol content on fenofibrate solubility in E463808. The solubility profile shows a non-obvious solubility enhancement with a maximum solubility between 10-40% ethanol (v/v). It is unique to observe this type of non-linearity in non-aqueous systems. The increase in fenofibrate solubility in the presence of ethanol was not limited only to E463808. Significant solubility increases were also observed with other omega-3 oils such as those with EPA:DHA ratios of 75:11, 10:50, and 45:10. The 75:11 oil comprises EPA and DHA in ethyl ester form while the 10:50 and 45:10 oils comprise EPA and DHA in triglyceride form.

### Example 2

### Fenofibrate Solubility in E463808-Based Formulations

### Temperature Dependence

It was noted that the solubility of the formulations of Example 1 showed a strong dependence on temperature. The experiment of this example studied this effect in greater detail.

Saturated fenofibrate samples were prepared under three controlled temperatures: 4° C., 23° C., and 33° C. After overnight stirring and incubation, the samples were filtered using a 0.2 micrometer PVDF syringe filter. The filter apparatus was pre-incubated at the sample temperature before use. The filtrates were promptly diluted and analyzed via normal phase HPLC.

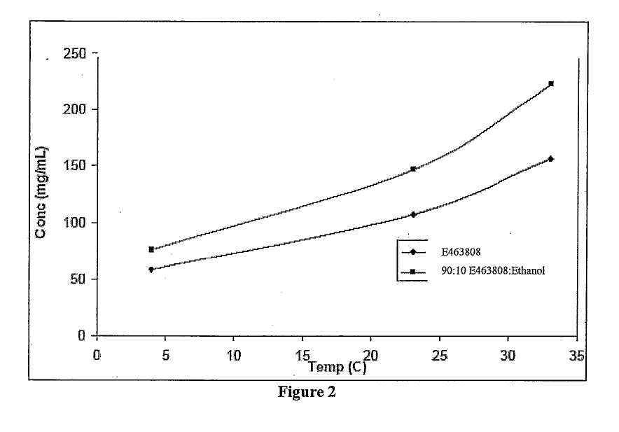

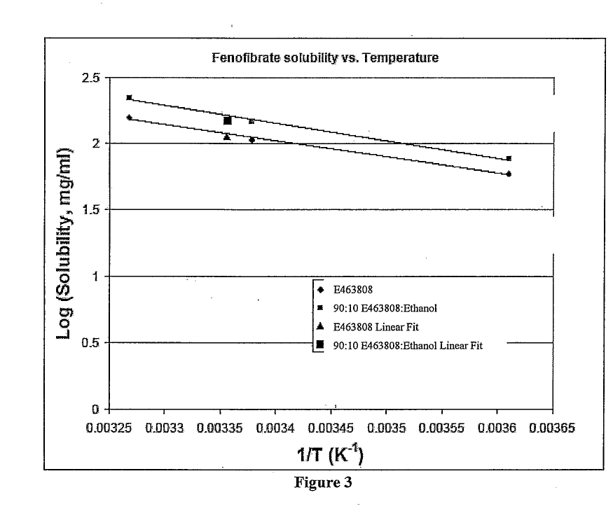

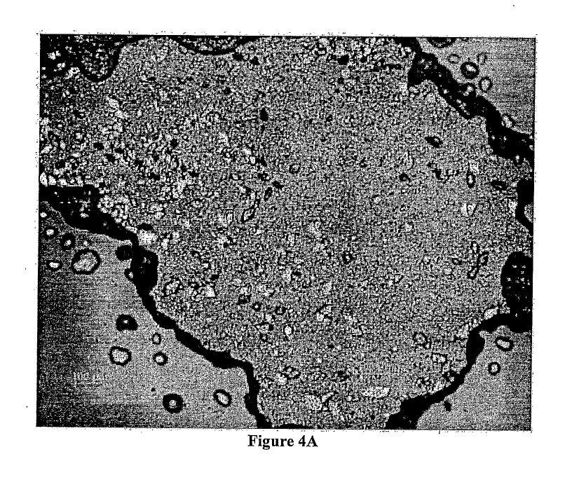

Fenofibrate solubility versus temperature in two vehicles (100% E463808, and 90:10 E463808:ethanol v/v) were measured and are illustrated in FIG. 2. The solubility of fenofibrate showed a relatively steep dependence on temperature. The Van't Hoff-type plot is illustrated in FIG. 3.

### Example 3

### Colloidal Suspensions and Nonionic Polymers

The objective of the experiment of this example was to identify additives that could induce crystal nucleation, which would result in smaller fenofibrate crystals from cold solutions that would redissolve more rapidly as temperature was increased. It was also an objective of the experiment to identify additives that would prevent fenofibrate crystals from adhering to one another and thereby decreasing surface area.

High-molecular weight ionic polymers may adsorb onto crystal surfaces and provide sufficient stability against aggregation or excessive growth. Orally-acceptable ionic polymers including Poly(vinyl acetate co-crotonic acid) (PVA), Cellulose acetate phthalate, Eudragit® L100 (enteric methacrylate polymer), Eudragit® RS100 (swellable methacrylate polymer), and Crospovidone (Crosslinked povidone) have been used extensively as enteric-coating materials.

Eudragit® L100 was used to induce crystal nucleation to create smaller fenofibrate crystals as follows.

Approximately 50 microliter of a 10 mg/mL solution of Eudragit® L100 in ethanol was combined with 450 microliter of a 180 mg/mL Fenofibrate solution (heated to 37 degrees C.) in 90:10 E463808:Ethanol (v/v). The solution was thoroughly mixed and filled into size 1 gelatin capsules. The capsules were stored in a glass vial at room temperature.

After incubation for 24 hours, a hazy, translucent, gel-like globule was observed at the bottom of the capsule. No crystals could be detected by eye. However, after 3-4 days, multiple large crystals began growing from inside the gel-like globule. The crystals that were inside the globule were white in appearance, while the globule itself was clear.

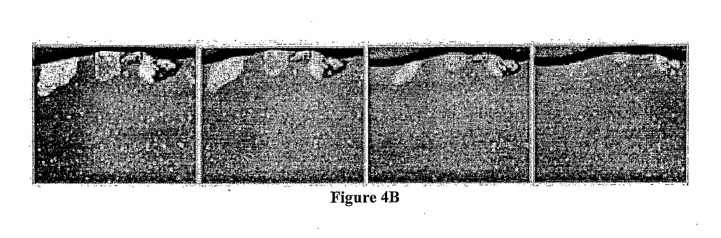

FIG. 4A illustrates polarized light microscopy of the gel-like globule. The globule consisted of approximately 100 micrometer fenofibrate crystals dispersed among micron-sized crystals in the polymer matrix. The polymer matrix alone without fenofibrate did not show any birefringence, and the PXRD pattern indicated an amorphous material.

When the temperature of the sample was raised to 45° C. via a hot stage to help facilitate the re-dissolution of the crystals, it was noticed that the larger crystals quickly dissolved, while the small micron-sized crystals remain unchanged. As illustrated in FIG. 4B, the globule was heated to 45° C. to facilitate re-dissolution of fenofibrate crystals. The larger crystals quickly dissolved, while the smaller particles remained.

A small portion of the fenofibrate in a liquid formulation may crystallize upon storage. However, re-solubilization of the majority of crystallized fenofibrate was achieved via inducing the growth of smaller crystals with Eudragit® polymer.

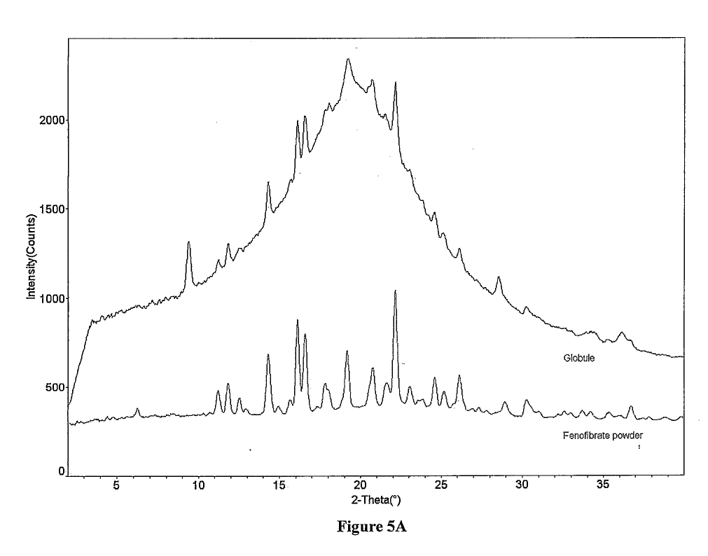

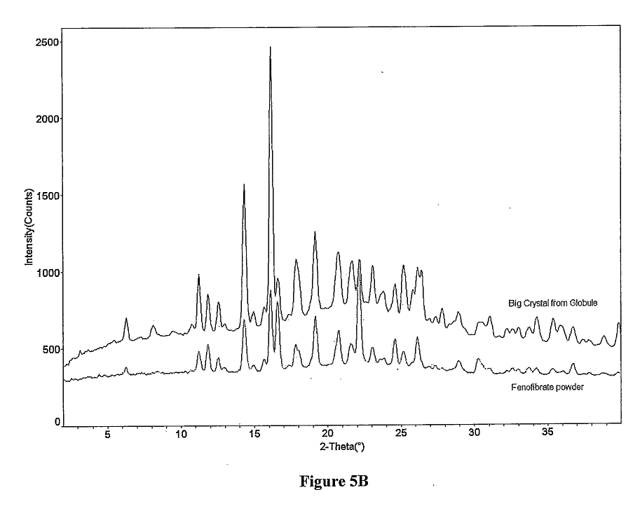

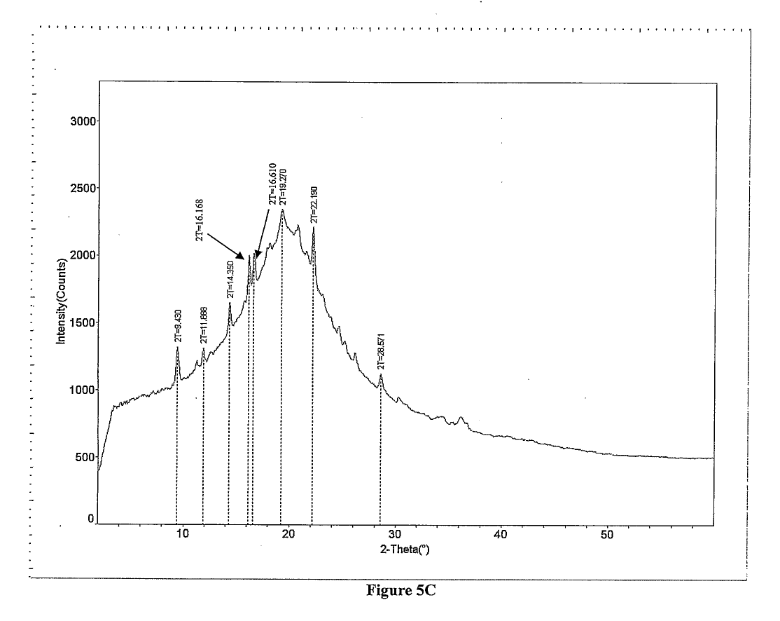

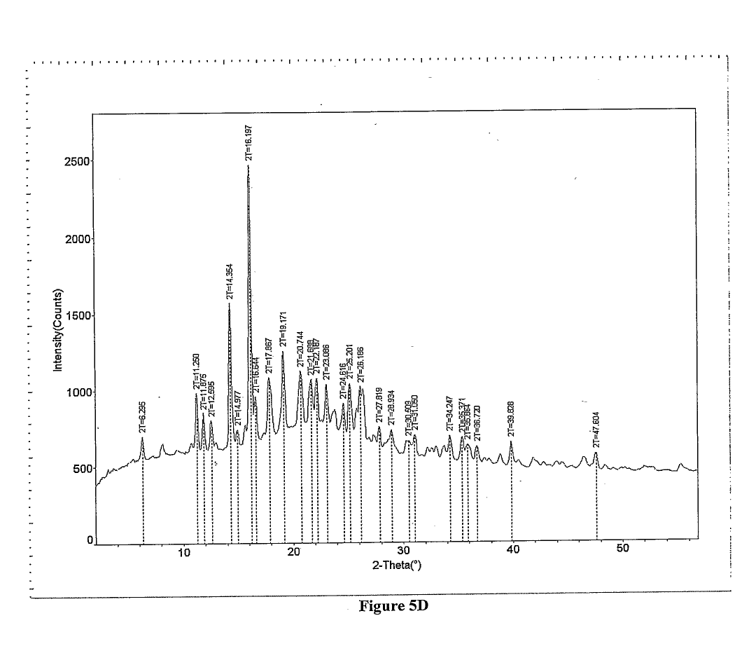

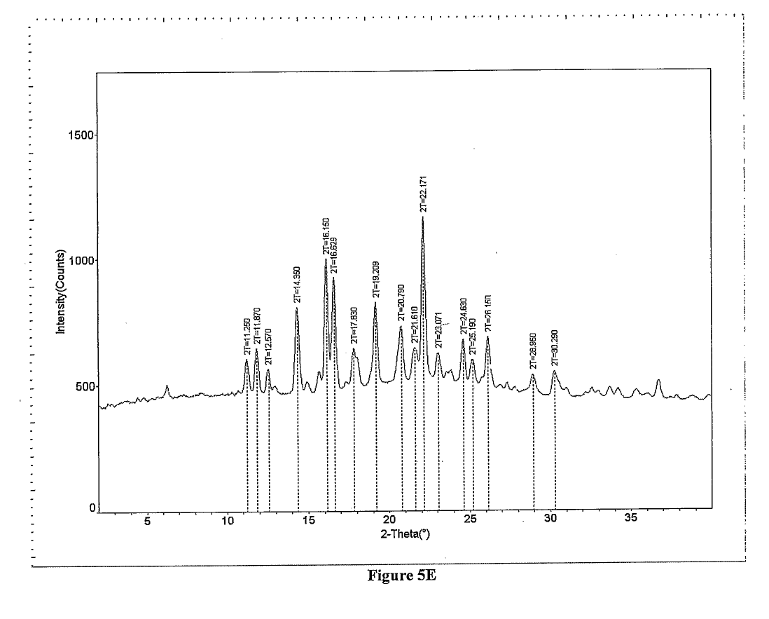

PXRD of samples of the fenofibrate-Eudragit® L100 globules exhibited identifying features that include those illustrated in FIG. 5A and listed below in Table 5.

TABLE 5
PXRD data of Fenofibrate-Eudragit L100 globules
Fenofibrate- Eudragit ® L100 globules
2-theta

9.43
11.89
14.35
16.17
16.61
19.27
22.19
28.57

The diffraction pattern illustrated in FIG. 5A and in Table 5 above indicated a large amount of amorphous material, which probably comprised the Eudragit® L100 polymer matrix with some crystalline content. FIG. 5C shows the labeled PXRD diffractogram of the globule data shown in FIG. 5A.

Samples of large fenofibrate crystals found inside the fenofibrate-Eudragit® L100 globules found were analyzed by PXRD. The samples exhibited powder X-ray diffraction pattern with identifying features that include those illustrated in FIG. 5B and listed below in Table 6. FIG. 5D shows the labeled PXRD diffractogram of the large crystal taken from the globule data shown in FIG. 5B.

TABLE 6
PXRD data of large fenofibrate crystal
from Fenofibrate-Eudragit L100 globules
Crystals from Fenofibrate- Eudragit ® L100 globules
2-theta
11.25
11.88
12.60
14.35
16.20
19.17
20.74
22.19
23.09

Table 7 and FIG. 5E show comparative PXRD data of fenofibrate (Form I) powder.

TABLE 7
PXRD data of Fenofibrate
Fenofibrate
2-theta
11.25
11.87
12.57
14.35
16.15
19.21
20.79
22.17
23.07

### Example 4

### Qualitative In Vitro Dissolution Experiment

Liquid formulations of the invention comprising fenofibrate were dispersed in various media at 37° C. to determine qualitative in vitro dissolution characteristics of the formulations.

The experimental setup consisted of a 20 mL sample vial in a 37° C. constant temperature water bath. The sample vial contains 15 mL of the desired aqueous medium: water, SGF (simulated gastric fluid), FaSSIF (fasted-state simulated intestinal fluid), or FeSSIF (fed-state simulated intestinal fluid), and a magnetic stir bar. FeSSIF consists essentially of 0.87 g acetic acid, 0.81 g sodium taurocholate, 0.295 g lecithin, 1.187 g sodium chloride, with a pH adjusted to 5.0 with sodium hydroxide and diluted to 100 mL with deionized water. FaSSIF consists essentially of 0.395 g NaH₂PO₄, 0.161 g of sodium taurocholate, 0.059 g of lecithin, 0.619 g sodium chloride, with a pH adjusted to 6.5 with sodium hydroxide and diluted to 100 mL with deionized water. SGF consists essentially of 1.0 g Triton X100, 2.0 g sodium chloride, with a pH adjusted to 2.0 with 1M HCl, and dissolved in 1000 mL distilled water. A 150 microliter aliquot of the desired formulation was added to the vial and gently stirred. Vials were briefly removed at certain time intervals and photographed.

Formulations 1-3 were subjected to this procedure and the results noted below were observed.

### Formulation 1

A 150 microliter aliquot of a 90:10 mixture (volume/volume) of E463808 and ethanol was mixed with fenofibrate in the weight percentages and amounts set forth in Table 8 below.

TABLE 8
Formulation 1 composition

| Formulation | Weight (mg) | Weight Percentage |
|---|---|---|
| E463808 | 724 | 77.6 |
| Ethanol | 69 | 7.4 |
| Fenofibrate | 140 | 15.0 |

The following observations were made with respect to formulation 1. The solubility of fenofibrate in the formulation was 152 mg/mL at 25° C. The solubility of the formulation in FaSSIF was 140 mg/ml. The formulation did not emulsify after 60 minutes and the formulation remained as an oil on the surface of the aqueous medium. Formulation 1 did not show any emulsification in water, SGF, and FeSSIF at 37° C. A pharmaceutical composition comprising this formulation may be appropriately administered to a patient in need of a therapeutic effect.

### Formulation 2

A 150 microliter aliquot of a 80:10:10 mixture (volume/volume) of E463808, ethanol, and Labrafac® CC (Gattefossè) medium chain triglyceride (C₈-C₁₀) carrier was mixed with fenofibrate in the weight percentages and amounts set forth in Table 9 below.

TABLE 9
Formulation 2 composition

| Formulation | Weight (mg) | Weight Percentage |
|---|---|---|
| E463808 | 630 | 68.5 |
| Ethanol | 68 | 7.4 |
| Labrafac ® CC | 82 | 9.0 |
| Fenofibrate | 140 | 15.1 |

The following observations were made with respect to formulation 2. The solubility of fenofibrate in the formulation was 137 mg/mL at 25° C. The formulation did not exhibit emulsification after 60 minutes and the formulation remained as an oil on the surface of the aqueous medium. Formulation 2 did not show any emulsification in water, SGF, and FeSSIF at 37° C. A pharmaceutical composition comprising this formulation may be appropriately administered to a patient in need of a therapeutic effect.

### Formulation 3

A 150 microliter aliquot of a 70:20:10 mixture (volume/volume) of E463808, ethanol, and Cremophor® EL (BASF) glycerol polyethylene glycolricinoleate-containing nonionic solubilizer and emulsifier was mixed with fenofibrate in the weight percentages and amounts set forth in Table 10 below.

TABLE 10
Formulation 3 composition

| Formulation | Weight Percentage |
|---|---|
| E463808 | 59 |
| Ethanol | 7.5 |
| Cremophor ® EL | 19.5 |
| Fenofibrate | 14 |

The following observations were made with respect to formulation 3. The solubility of the formulation in water was 140 mg/ml. Initially, the formulation appeared as a clear fluid solution which floated on the surface of the medium. After five minutes, most of the formulation had emulsified. There was a large distribution of droplets having an average size of around 300 nm. A pharmaceutical composition comprising this formulation may be appropriately administered with or without food.

### Example 5

### In Vitro Lipid Digestion Study

The properties of a formulation comprising fenofibrate was evaluated under conditions which simulated in vivo lipid digestion. The formulation consisted of between about 82-86 mg/mL fenofibrate dissolved in E463808.

Method: Subject to the variations noted herein, the protocols described in Sek et. al. (J. Pharm. Biomed. Anal. 25 (2001) 651-661) were applied in the experiment of this example.

Briefly, the digestion experiment was conducted in a 30 mL glass vial. A water bath was used to keep the sample temperature at 37° C. A pH probe measured the sample pH, and a manual titrator filled with 0.2 M sodium hydroxide was used to keep the sample pH at 7.5. The vial was initially filled with 20 mL of digestion buffer. The sample was then added, followed by 1 mL of pancreatic extract. The digestion was allowed to proceed for approximately 60 minutes. During that time, 1 mL samples were taken at various time points (5, 10, 15, 30, 45, and 60 minutes) and were carefully filtered with a 0.2 micrometer PVDF syringe filter to collect the aqueous phase. The samples were then analyzed via HPLC. At the end of the digestion, the vial contents were transferred to a 15 mL centrifuge tube and centrifuged at 5500 rpm for 35 minutes at 37° C. to separate the sample mixture into 3 phases—undigested oil on top, the middle aqueous phase, and the bottom solid phase.

After the centrifugation, the aqueous phase was isolated, filtered, and submitted for fenofibrate content determination via HPLC. The solid phase was extracted with acetonitrile to capture any precipitated fenofibrate and the captured fenofibrate was also analyzed by HPLC.

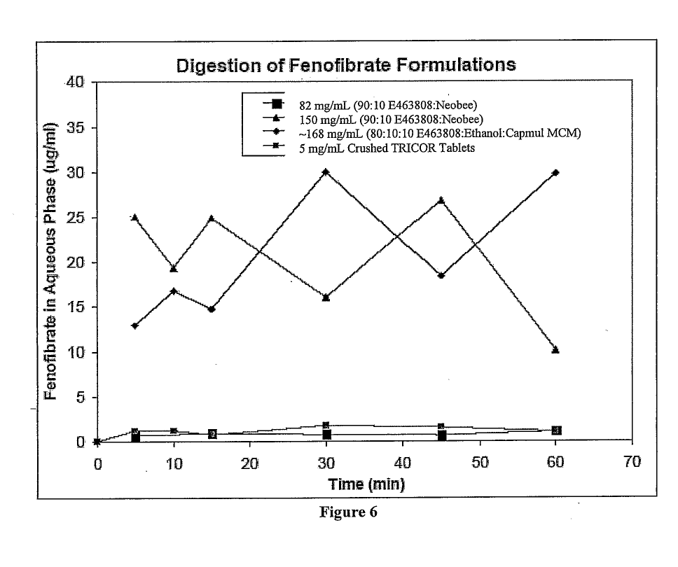

Results: It was observed that the majority of fenofibrate remained solubilized in the oil phase in the formulation. We found that an increase in drug partitioning out of the oil phase and into the aqueous phase could be achieved by blending in a small amount of Neobee M5 or Capmul MCM without significant precipitation, as illustrated in FIG. 6. In addition, it was observed that formulations with a higher drug loading increased the partitioning of fenofibrate from the vehicle into the aqueous phase.

### Example 6

### Stability and Compatibility of Fenofibrate and E463808

Table 11 below sets forth conditions of prepared samples for stability studies of fenofibrate in E463808 under various conditions. In all cases, no degradation of fenofibrate could be detected after up to eight weeks of storage. The fenofibrate stability samples along with placebos (i.e., E463808 stored at the same condition and for the same time period) were run using two separate gradient HPLC methods. The chromatograms of each sample were overlaid with those of the corresponding placebo to detect the presence of any peaks unrelated to E463808 and therefore due to fenofibrate degradation. Upon analysis of the samples stored under the conditions described in Table 11 for up to 8 weeks, no degradation was observed.

TABLE 11
HPLC degradation data of Fenofibrate

| Conc. | | 4 deg | 25 deg C./ | 40 deg C./ | |
|---|---|---|---|---|---|
| (mg/mL) | Enclosure | C. | 60% RH | 75% RH | 60 deg C. |
| 80 | sealed vial | | X | X | X |
| 200 | sealed vial | | X | X | X |
| 80 | gelcap | X | X | | |
| 200 | gelcap | X | X | | |

### Example 7

### Solubility of Fenofibrate in E463808-Additive Mixtures

Saturated solutions of fenofibrate in E463808 and a second additive were prepared at a controlled temperature of 25° C. Equilibrated samples were filtered using 0.2 micrometer PVDF syringe filters and diluted for assay. The fenofibrate and E463808 solutions included additives selected from the list consisting of: Tween 85, Tween 80, Cremophor EL, Span 80, Span 85, and ethanol. (Tween 85 is also known as polyoxyethylenesorbitan trioleate, Tween 80 is also known as polyoxyethylenesorbitan monooleate, Span 80 is also known as sorbitan monooleate, Span 85 is also known as sorbitan trioleate, and Cremophor EL is also known as polyoxyl castor oil.) Span 80, Span 85, and Tween 85 samples were analyzed using normal-phase HPLC. Cremophor EL, Tween 80, and replicates of Tween 85 samples were analyzed using UV measurements at 285 nm. The latter set of samples required UV analysis due to their lack of miscibility with the mobile phase (heptane).

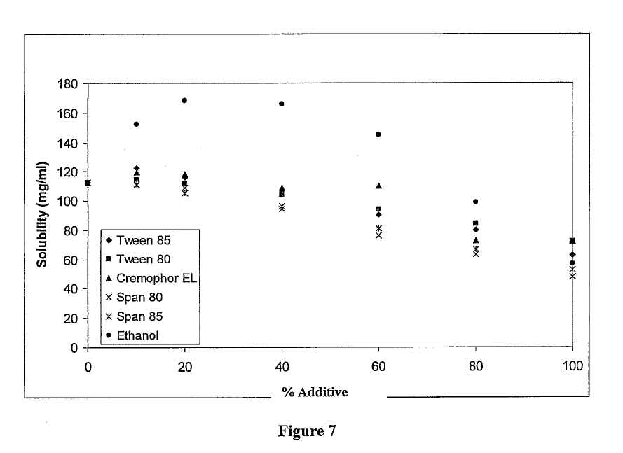

FIG. 7 illustrates the solubility of fenofibrate in E463808-additive mixtures (% v/v) at 25° C.

E463808 was immiscible with Cremophor® EL (with Cremophor® EL at less than approximately 50% v/v), and perhaps Span 80® (Span 80® itself had a hazy appearance). Adding 9-10% v/v ethanol resulted in miscible mixtures. Slight enhancement in fenofibrate solubility at about 10% v/v additive was observed with PEG-containing surfactants such as Tween 85®, Tween 80® and Cremophor® EL. Combining E463808 with more hydrophobic surfactants (Spans) decreased fenofibrate solubility (The Span® brand have the same number of hydroxyl groups as Tweens (3) but are smaller in size). However, the degree of increased solubility was much more evident in E463808/Ethanol mixtures. Based on these data, it appears that including approximately 10% v/v ethanol not only helps to increase fenofibrate solubility, but it also creates miscible formulations of E463808 and certain surfactants.

FIG. 7 shows a solubility enhancement of fenofibrate in E463808 with ethanol (about 170 mg/mL at 20% ethanol v/v) that is much greater than the enhancement of fenofibrate solubility in E463808 with any surfactant (about 120 mg/mL at 20% surfactant v/v). Because of this, liquid formulations of the invention comprise an alcohol to facilitate larger doses of fenofibrate. Surfactants may be added to liquid formulations in order to increase in vivo bioavailability in the fasted state, but do not provide sufficient solubilization power to significantly increase the fenofibrate dosage.

### Example 8

### Equilibrium Fenofibrate Solubility in E463808/Ethanol/Surfactant Combinations

Tables 12 and 13 show the solubility of fenofibrate in various liquid formulations comprising E463808 at 25 degrees C.

TABLE 12
Fenofibrate solubility at 25 degrees C. in E463808
formulations with Ethanol and Tween 80 (v/v)

| E463808 | Ethanol | Tween 80 | Solubility |
|---|---|---|---|
| (v/v) | (v/v) | (v/v) | (mg/mL) |
| 85 | 5 | 10 | 137 |
| 75 | 15 | 10 | 151 |
| 70 | 10 | 20 | 136 |
| 60 | 20 | 20 | 142 |
| 55 | 15 | 30 | 132 |
| 65 | 5 | 30 | 117 |
| 50 | 10 | 40 | 124 |
| 40 | 20 | 40 | 131 |
| 100 | 0 | 0 | 113 |
| 95 | 5 | 0 | 135 |
| 90 | 10 | 0 | 152 |
| 85 | 15 | 0 | 162 |
| 80 | 20 | 0 | 168 |
| 90 | 0 | 10 | 114 |
| 80 | 0 | 20 | 112 |
| 70 | 0 | 30 | 108 |
| 60 | 0 | 40 | 104 |

The highest solubility (168.0 mg/mL) is reached with an 80:20 E463808:ethanol mixture.

TABLE 13
Fenofibrate solubility at 25 degrees C. in E463808
formulations with ethanol and Cremophor EL (v/v)

| E463808 | Ethanol | Cremophor | Solubility |
|---|---|---|---|
| (v/v) | (v/v) | EL (v/v) | (mg/mL) |
| 85 | 5 | 10 | 132 |
| 75 | 15 | 10 | 149 |
| 70 | 10 | 20 | 141 |
| 60 | 20 | 20 | 142 |
| 55 | 15 | 30 | 131 |
| 65 | 5 | 30 | 124 |
| 50 | 10 | 40 | 124 |
| 40 | 20 | 40 | 127 |
| 100 | 0 | 0 | 113 |
| 95 | 5 | 0 | 135 |
| 90 | 10 | 0 | 152 |
| 85 | 15 | 0 | 162 |
| 80 | 20 | 0 | 168 |
| 90 | 0 | 10 | 119 |
| 80 | 0 | 20 | 118 |
| 70 | 0 | 30 | 114 |
| 60 | 0 | 40 | 109 |

### Example 9

### Equilibrium Fenofibrate Solubility in Ethyl Esters Versus Triglycerides

Table 14 shows a comparison of fenofibrate solubility in ethyl esters and that in corresponding triglycerides at 25 degrees C. Polarity and the number of C═C double bonds correlate with increased fenofibrate solubility. Importantly, fenofibrate shows higher solubility consistently in ethyl esters than in a corresponding triglyceride.

TABLE 14
Fenofibrate solubility at 25 degrees C.
in ethyl esters and in triglycerides

| Vehicle | Description | Solubility (mg/mL) |
|---|---|---|
| Ethyl caprylate | C8, ethyl ester | 177.8 |
| Ethyl caprate | C10, ethyl ester | 142.2 |
| Neobee M5 | C8 and C10, triglyceride | 82.0 |
| Ethyl oleate | C18, 1 double bond, ethyl ester | 86.7 |
| Triolein | C18, 1 double bond, triglyceride | 48.9 |
| Ethyl linoleate | C18, 2 double bonds, ethyl ester | 92.3 |
| Trilinolein | C18, 2 double bonds, | 61.2 |
| | triglyceride | |

### Example 10

### Determination of Increased Solubilization Power with Ethanol and Ethyl Esters

A saturated solution of fenofibrate (125.80 mg) in TG361724 fish oil was prepared by adding the fish oil to the fenofibrate up to a volume of 1 mL. The fish oil was comprised of triglycerides. A stir bar was added and the container was crimp sealed. The container was placed in a water bath at 25 degrees C. and stirred overnight. The sample was then filtered through a 0.2 micrometer PVDF syringe filter, the liquid was collected and diluted in ethanol by a factor of 2000. A UV spectrophotometer (285 nm) was used to measure the fenofibrate concentration. The solubility of fenofibrate in pure TG361724 is reported below in Table 15.

A saturated solution of fenofibrate (145.47 mg) in a 90:10 solution by volume of TG361724:ethanol was prepared by adding the fish oil:ethanol mixture to the fenofibrate up to a volume of 1 mL. The fish oil was comprised of triglycerides. A stir bar was added and the container was crimp sealed. The container was placed in a water bath at 25 degrees C. and stirred overnight. The sample was then filtered through a 0.2 micrometer PVDF syringe filter, the liquid was collected and diluted in ethanol by a factor of 2000. A UV spectrophotometer (285 nm) was used to measure the fenofibrate concentration. The solubility of fenofibrate in a mixture of 90:10 TG361724:ethanol is reported below in Table 15.

A saturated solution of fenofibrate (125.46 mg) in E351923 was prepared by adding the fish oil to the fenofibrate up to a volume of 1 mL. The fish oil was comprised of ethyl esters. A stir bar was added and the container was crimp sealed. The container was placed in a water bath at 25 degrees C. and stirred overnight. The sample was then filtered through a 0.2 micrometer PVDF syringe filter, the liquid was collected and diluted in ethanol by a factor of 2000. A UV spectrophotometer (285 nm) was used to measure the fenofibrate concentration. The solubility of fenofibrate in pure E351923 is reported below in Table 15.

A saturated solution of fenofibrate (201.74 mg) in a 90:10 solution by volume of E351923:ethanol was prepared by adding the fish oil:ethanol mixture to the fenofibrate up to a volume of 1 mL. The fish oil was comprised of ethyl esters. A stir bar was added and the container was crimp sealed. The container was placed in a water bath at 25 degrees C. and stirred overnight. The sample was then filtered through a 0.2 micrometer PVDF syringe filter, the liquid was collected and diluted in ethanol by a factor of 2000. A UV spectrophotometer (285 nm) was used to measure the fenofibrate concentration. The solubility of fenofibrate in a mixture of 90:10 E351923:ethanol is reported below in Table 15.

TABLE 15
Fenofibrate solubility in several oils and
oil:ethanol mixtures at 25 degrees C.

| Liquid Vehicle | Solubility (mg/mL) |
|---|---|
| TG361724 | 67.3 |
| 90:10 TG361724:ethanol | 88.5 |
| E351923 | 95.6 |
| 90:10 E351923:ethanol | 129.0 |

A saturated solution of fenofibrate (130.9 mg) in E107104 was prepared by adding the fish oil to the fenofibrate up to a volume of 1 mL. The fish oil was rich in DHA. A stir bar was added and the container was crimp sealed. The container was placed in a water bath at 25 degrees C. and stirred overnight. The sample was then filtered through a 0.2 micrometer PVDF syringe filter, the liquid was collected and diluted in ethanol by a factor of 2000. A UV spectrophotometer (285 nm) was used to measure the fenofibrate concentration. The solubility of fenofibrate in pure E107104 is reported below in Table 16.

A saturated solution of fenofibrate (151.3 mg) in a 95:5 solution by volume of E107104:ethanol was prepared by adding the fish oil:ethanol mixture to the fenofibrate up to a volume of 1 mL. The fish oil was rich in DHA. A stir bar was added and the container was crimp sealed. The container was placed in a water bath at 25 degrees C. and stirred overnight. The sample was then filtered through a 0.2 micrometer PVDF syringe filter, the liquid was collected and diluted in ethanol by a factor of 2000. A UV spectrophotometer (285 nm) was used to measure the fenofibrate concentration. The solubility of fenofibrate in a mixture of 95:5 E107104:ethanol is reported below in Table 16.

A saturated solution of fenofibrate (161.6 mg) in a 90:10 solution by volume of E107104:ethanol was prepared by adding the fish oil:ethanol mixture to the fenofibrate up to a volume of 1 mL. The fish oil was rich in DHA. A stir bar was added and the container was crimp sealed. The container was placed in a water bath at 25 degrees C. and stirred overnight. The sample was then filtered through a 0.2 micrometer PVDF syringe filter, the liquid was collected and diluted in ethanol by a factor of 2000. A UV spectrophotometer (285 nm) was used to measure the fenofibrate concentration. The solubility of fenofibrate in a mixture of 90:10 E107104:ethanol is reported below in Table 16.

A saturated solution of fenofibrate (154.2 mg) in E970002 was prepared by adding the fish oil to the fenofibrate up to a volume of 1 mL. The fish oil was rich in EPA. A stir bar was added and the container was crimp sealed. The container was placed in a water bath at 25 degrees C. and stirred overnight. The sample was then filtered through a 0.2 micrometer PVDF syringe filter, the liquid was collected and diluted in ethanol by a factor of 2000. A UV spectrophotometer (285 nm) was used to measure the fenofibrate concentration. The solubility of fenofibrate in pure E970002 is reported below in Table 16.

A saturated solution of fenofibrate (204.8 mg) in a 90:10 solution by volume of E970002:ethanol was prepared by adding the fish oil:ethanol mixture to the fenofibrate up to a volume of 1 mL. The fish oil was rich in EPA. A stir bar was added and the container was crimp sealed. The container was placed in a water bath at 25 degrees C. and stirred overnight. The sample was then filtered through a 0.2 micrometer PVDF syringe tilter, the liquid was collected and diluted in ethanol by a factor of 2000. A UV spectrophotometer (285 nm) was used to measure the fenofibrate concentration. The solubility of fenofibrate in a mixture of 90:10 E970002:ethanol is reported below in Table 16.

TABLE 16
Fenofibrate solubility in several EPA and DHA-rich
oils and oil:ethanol mixtures at 25 degrees C.

| Liquid Vehicle | Solubility (mg/mL) |
|---|---|
| E107104 | 102.2 |
| 95:5 E107104:ethanol | 124.5 |
| 90:10 E107104:ethanol | 132.1 |
| E970002 | 106.7 |
| 90:10 E970002:ethanol | 140.8 |

Table 15 shows an increased solubility of fenofibrate in omega-3 oils when ethanol is added to the formulation. Although this increase is seen in omega-3 triglyceride-based oils as well as omega-3 ethyl ester-based oils, it is only the ethyl ester-based omega-3 oils that provide the fenofibrate solubility at and above 100 mg/mL which is necessary for liquid formulations of the present invention. Table 16 shows a similar increase in fenofibrate solubility with the addition of ethanol. Also, omega-3 oils with a high content of DHA and omega-3 oils with a high content of EPA both provide similar solubilization power. Based on the above data, the ratio of EPA:DHA does not appear to be a critical variable for the increased solubilization power of fenofibrate in omega-3 oil.

### Example 11

### Fenofibrate Polymorph

### Form II

In a 5 mL glass vial, 500.3 mg of Gelucire® 44/14 and 500.3 mg of poloxamer 407 were dispensed and continuously mixed with a magnetic stir-bar. The mixture was heated in a water bath to 85 degrees C. until all components were molten. 670.1 mg of fenofibrate was slowly added and mixed for an additional 20 minutes. The temperature was reduced to 70 degrees C. and mixed for another 20 minutes. 50 microliters of this sample was collected and placed into a glass vial that had been prepared at 70 degrees C. The glass vial was immediately cooled by placement into an acetone/dry ice bath and then placed at 4 degrees C. A solid formed and was collected for PXRD analysis. The solid was determined to be a fenofibrate polymorph (Form II).

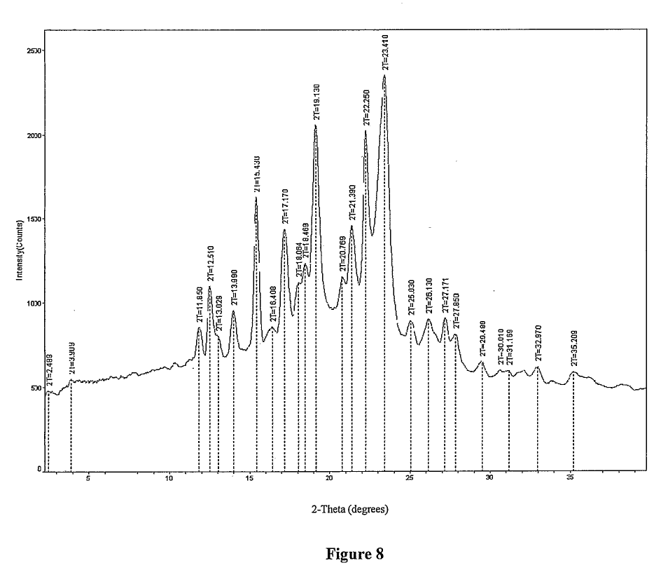

PXRD diffractogram for the fenofibrate polymorph (Form II) is shown in FIG. 8. The fenofibrate polymorph (Form II) can be characterized by any one, any two, any three, any four, any five, or any six or more of the peaks in FIG. 8 including, but not limited to, 11.85, 12.51, 13.99, 15.43, 17.17, 18.47, 19.13, 21.39, 22.25, 23.41, 25.03, 26.13, and 27.17 degrees 2-theta (Rigaku, data as collected).

The fenofibrate polymorph (Form II) was also prepared in pure Gelucire® 44/14 and in pure poloxamer 407.

### Example 12

### Fenofibrate Formulation

A formulation was prepared and is described in Table 17, below. The fenofibrate was completely soluble in the formulation. The fenofibrate solubility was determined to be 87.9 mg/mL (+/−˜5 mg/mL) at 25 degrees C. via the UV absorption method described previously.

TABLE 17
Formulation of Fenofibrate

| Formulation | Quantity (mg)* |
|---|---|
| EPAX ® 4510 TG | 189 |
| E463808 | 95 |
| Tween 80 | 136 |
| Myrj 52 | 236 |
| Vitamin E TPGS | 164 |
| Labrasol | 127 |
| Ethanol | 91 |
| Propylene glycol | 182 |
| Fenofibrate | 100 |

*= volume weighted

### Example 13

### Pharmacokinetic Analysis of Fenofibric Acid in Humans

The pharmacokinetics of fenofibric acid were evaluated in humans. 18 healthy subjects (male and female) were selected for this study. The study design was a single-dose, 3 treatment, and 3-sequence, 3-period crossover with a washout interval of at least one-week between each period. An equal number of subjects (i.e. six) was randomly assigned to each of the three sequences. Following an overnight fast of at least 10 hours, subjects were given a single dose of the following test or reference treatment with 240 mL of water:

1. Fenofibrate/omega-3, 160 mg capsule after a standard breakfast;
2. Fenofibrate/omega-3, 160 mg capsule after an overnight fast; and
3. Tricor®, 160 mg tablet after a standard breakfast;
wherein the fenofibrate/omega-3 administered formulation comprised the components and amounts shown in Table 18.

TABLE 18
Fenofibrate/omega-3 Formulation Administered to Humans

| Component | Weight percent | Per dose (mg) (2 capsules) |
|---|---|---|
| Fenofibrate | 15.11 | 160.00 |
| E681010 | 73.69 | 780.16 |
| Ethanol | 11.20 | 118.57 |

Venous blood samples were collected pre-dose (0 hours) and 1, 2, 3, 4, 6, 8, 10, 14, 24, 34, 48 and 72 hours post-dose. Plasma from the collected blood samples were promptly separated and frozen until assayed using a validated assay for fenofibric acid in human plasma with a lower limit of quantitation of 20.1 ng/mL.

The pharmacokinetic measures, including AUC₀₋ₜ, AUC_{0-inf}, Cₘₐₓ, Tₘₐₓ and t½ were calculated from the individual concentration-time data for fenofibric acid using PhAST software (Phoenix international). Analysis of variance (ANOVA) was performed for log-transformed data of AUC₀₋ₜ, AUC_{0-inf}, and Cₘₐₓ. In Table 19 below, “Cₘₐₓ”″ is the maximum blood plasma concentration, “AUC₀₋ₜ” is the area under the curve from time point 0 to 72 hours post-dose, “AUC_{0-inf}” is the extrapolated area under the curve, “t_{1/2}” is the amount of time for the blood plasma level to decrease to half of the Cₘₐₓ level beginning at administration, “Tₘₐₓ” is the time to maximum blood plasma concentration from administration, and “F” is the percent bioavailability.

TABLE 19
Summary of Mean (SD) Pharmacokinetic Parameters of Fenofibric Acid in Humans
Following Oral Administration of Two Formulations of Fenofibrate

| | AUC₀₋ₜ | AUC_{0-inf} | Cₘₐₓ | Half-life | Tmax | F | F |
|---|---|---|---|---|---|---|---|
| Treatment | (ng/mL × hr) | (ng/mL × hr) | (ng/mL) | (hr) | (hr) | (AUC₀₋ₜ₎ | (AUC_{0-inf}) |
| Tricor 160 | 167728 | 182132 | 9899 | 19.2 | 5.94 | NA | NA |
| mg fed | (43953.8) | (55371.7) | (3075.1) | (5.65) | (7.62) | | |
| Fenofibrate/ | 175534 | 189497 | 8719 | 17.7 | 10.0 | 105.0 | 104.1 |
| Omega-3 | (51051.4) | (60123.0) | (2438.4) | (4.85) | (7.06) | (20.6) | (16.1) |
| 160 mg fed | | | | | | | |
| Fenofibrate/ | 110032^{a} | 166326 | 3559^{a} | 37.1 | 15.8 | 67.2 | 87.9 |
| omega-3 | (58220.0) | (104979.1) | (3421.4) | (29.2) | (12.73) | (37.2) | (39.4) |
| 160 mg | | | | | | | |
| fasted | | | | | | | |

^{a}Statistically significant difference (p < 0-05) compared to Tricor ®

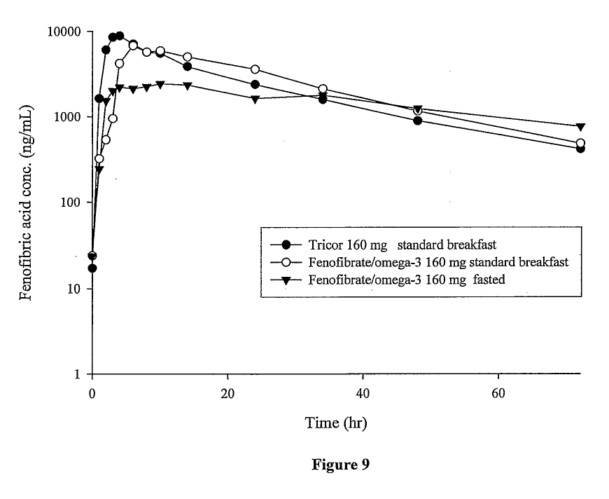

FIG. 9 shows a semi-log plot of mean plasma concentration of fenofibric acid in humans following oral administration of two formulations of fenofibrate.

### Example 14

### Fenofibrate Solubility in Various Oils at 15 Degrees C.

Table 20 shows the solubility of fenofibrate measured at 15 degrees C. in several oils and in several oil/ethanol mixtures.

TABLE 20
Solubility of fenofibrate in several vehicles at 15 degrees C.

| | Solubility |
|---|---|
| Vehicle | mg/mL |
| 85/15 wt % Captex ® 200/Ethanol | 126 |
| Captex ® 200 | 93 |
| 50/50 wt % Myvacet ® 9-45K/E681010 | 92 |
| E681010 | 90 |
| Myvacet ® 9-45K | 90 |
| 85/15 wt % Crodamol EO/Ethanol | 89 |
| Triomega ® Omega-3 | 85 |
| 85/15 wt % Eumulgin ® 05/Ethanol | 69 |
| 85/15 wt % Oleic Acid/Ethanol | 65 |
| Crodamol EO | 64 |
| 85/15 wt % Campul ® MCM/Ethanol | 63 |
| Epax ® 4510TG | 61 |
| Epax ® 1050TG | 60 |
| 85/15 wt % Peceol/Ethanol | 51 |
| FlaxSeed Oil | 47 |
| Cod Liver Oil | 43 |
| Oleic Acid | 39 |

In the table above and throughout the disclosure, Captex® 200 is also known as propylene glycol dicaprylate/dicaprate, Myvacet® 9-45K is also known as acetylated monoglycerides, Crodamol EO is also known as ethyl oleate, Capmul® MCM is also known as capric/caprylic glycerides, Peceol is also known as glycerol oleate, Epax® 4510TG is a concentrate containing 45 percent EPA and 10 percent DHA (triglycerides), Epax® 1050TG is a concentrate containing 10 percent EPA and 50 percent DHA (triglycerides), and Eumulgin® 05 is also known as ethocylated oleyl cetyl alcohol.

### Example 15

### Fenofibrate Solubility as a Function of Ethanol Concentration

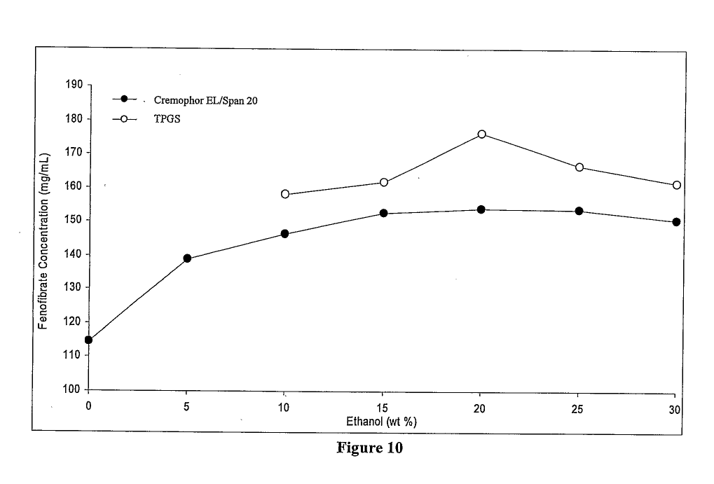

The solubility of fenofibrate was studied in two surfactant-containing formulations as a function of ethanol concentration. Formulation one comprised E681010:ethanol:Cremophor EL:Span 20, wherein the weight percent of Cremophor EL and Span 20 were each maintained at 10 percent. (For example, the samples contained component weight ratios of 80:0:10:10, 75:5:10:10, 70:10:10:10, 65:15:10:10, 60:20:10:10, 55:25:10:10, and 50:30:10:10.) Note, Span 20 is also known as sorbitan monolaurate. Formulation two contained E681010:ethanol:TPGS, wherein the weight percent of TPGS was maintained at 20 percent. (For example, the samples contained component weight ratios of 70:10:20, 65:15:20, 60:20:20, 55:25:20, and 50:30:20.) Note, TPGS is also known as d-alpha-tocopheryl polyethylene glycol 1000 succinate. FIG. 10 shows the data from zero percent to 30 percent ethanol by weight.

### Example 16

### Fenofibrate Solubility as a Function of Temperature

Formulation components were weighed and mixed to form homogeneous solutions. Excess fenofibrate was added to 1 mL of the premixed formulation into 10 mL vials. A stir bar was added and the vials were crimped. The formulations were incubated at fixed temperatures (e.g., 15, 25, 32° C.) using a circulating water bath for 24 to 72 hours under constant mixing. Post incubation, 1 mL of each mixture was filtered via syringe with a 0.45 micrometer pore size, 13 mm, PTFE filter. 50 to 100 microliters of the filtered solution was collected and diluted 1000-fold in volumetric flask with 30/70 v/v acetonitrile-water. Diluted samples were analyzed for fenofibrate content using HPLC with UV detection.

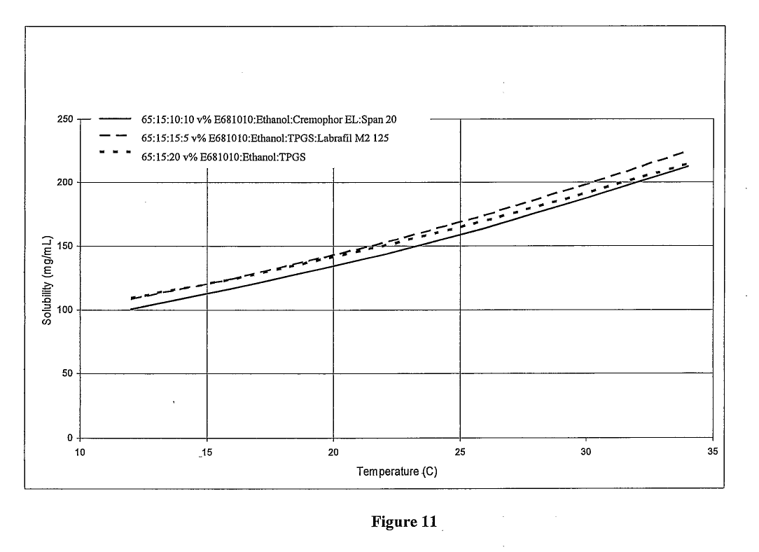

FIG. 11 shows the temperature dependence of fenofibrate solubility for three different formulations. The first formulation comprises E681010:ethanol:CremophorEL:Span 20 in a ratio of 65:15:10:10. The second formulation comprises E681010:ethanol:TPGS:Labrafil M2125 in a ratio of 65:15:15:5. The third formulation comprises E681010:ethanol:TPGS in a ratio of 65:15:20. Note, Labrafil M2125 is also known as linoleoyl polyoxylglycerides.

### Example 17

### Characterization of Emulsification Behavior

Filtered samples from the solubility studies were also used to characterize the emulsification behavior of several formulations. Fenofibrate was saturated in these samples. In the study, 64 microliters of formulation was added to a 20 mL solution of 34.2 mM sodium chloride in deionized water. (This simulates the addition of 0.8 mL gelatin capsule to a 250 mL resting stomach volume of fluid.) The sodium chloride solution represents simulated gastric fluid in the absence of a surfactant wetting agent. Observations to the emulsification process were: 1) Degree of Emulsification (in order of decreasing degrees): microemulsion, coarse emulsion, partial emulsion, poor emulsion, or no emulsification (none); and 2) Dispersion Speed: fast or slow.

Table 21 shows several surfactant-containing formulations of fenofibrate in E681010 and ethanol with variable ratios of oil, ethanol, and surfactant. The solubility measurement described in Table 21 were taken at 27 degrees C. The emulsification classification was completed at 37 degrees C. Note: All reports of weight percent in Table 21 are rounded to the nearest whole number, and therefore may include approximations of up to +/−0.5 percent by weight.

TABLE 21
Fenofibrate Solublity and Emulsification Data

| | Average | | |
|---|---|---|---|
| | Solubility | Degree of | Dispersion |
| Formulation Composition* | (mg/mL) | Emulsification | Speed |
| 100 wt % E681010 | 126 | None | N/A |
| 89/11 wt % E681010/Ethanol | 180 | None | N/A |
| 66/12/22 wt % E681010/Ethanol/Cremophor EL | 158 | Poor | Fast |
| 52/8/21/19 wt % E681010/Ethanol/Cremophor EL/Span20 | 139 | Coarse | Fast |
| 65/12/11/9/3 wt % E681010/Ethanol/Cremophor EL/ | 153 | None | N/A |
| Span20/Ethanolamine | | | |
| 67/13/11/10 wt % E681010/Ethanol/CremophorEL/ | 167 | Coarse | Fast |
| Crill 1 NF | | | |
| 70/11/9/10 wt % E681010/Ethanol/Cremophor EL/ | 174 | Poor | Slow |
| Alcolec EM | | | |
| 67/13/11/10 wt % | 161 | Coarse | Fast |
| E681010/Ethanol/CremophorEL/Span20 | | | |
| 66/13/11/9 wt % E681010/Ethanol/CremophorEL/Span80 | 154 | Partial | Fast |
| 66/13/11/10 wt % E681010/Ethanol/CremophorEL/ | 151 | Poor | Slow |
| Labrafil M2125 CS | | | |
| 67/12/11/10 wt % | 156 | Poor | Slow |
| E681010/Ethanol/CremophorEL/Labrasol | | | |
| 66/12/10/12 wt % | 149 | Poor | Slow |
| E681010/Ethanol/CremophorEL/Polysorbate 20 | | | |
| 65/16/1574 wt % E681010/Ethanol/Cremophor EL/ | 168 | Partial | Fast |
| Span20 | | | |
| 66/13/16/6 wt % E681010/Ethanol/CremophorEL/Span80 | 148 | DS | Fast |
| 67/13/15/5 wt % E681010/Ethanol/CremophorEL/ | 145 | Poor | Fast |
| Labrafil M2125 CS | | | |
| 67/13/15/6 wt % E681010/Ethanol/Cremophor/Labrasol | 147 | Poor | Fast |
| 67/13/2/19 wt % E681010/Ethanol/Cremophor EL/ | 169 | None | N/A |
| Span 20 | | | |
| 67/12/6/15 wt % E681010/Ethanol/Cremophor EL/ | 167 | Partial | Fast |
| Span 20 | | | |
| 67/13/6/15 wt % E681010/Ethanol/CremophorEL/ | 151 | Poor | Fast |
| Labrafil M2125 CS | | | |
| 66/12/6/16 wt % | 152 | None | N/A |
| E681010/Ethanol/CremophorEL/Labrasol | | | |
| 66/12/6/15 wt % | 147 | Poor | Slow |
| E681010/Ethanol/CremophorEL/Polysorbate 20 | | | |
| 66/13/11/10/1 wt % | 166 | Partial | Fast |
| E681010/Ethanol/CremophorEL/Span20/Glycocholic Acid | | | |
| 69/13/16/2 w % E681010/Ethanol/CremophorEL/ | 143 | Partial | Slow |
| Glycocholic Acid | | | |
| 66/8/26 wt % E681010/Ethanol/TPGS | 145 | Partial | Slow |
| 66/13/21 wt % E681010/Ethanol/TPGS | 164 | Partial | Slow |
| 76/8/16 wt % E681010/Ethanol/TPGS | 158 | Poor | Slow |
| 77/12/10 wt % E681010/Ethanol/TPGS | 178 | Poor | Fast |
| 76/4/20 wt % E681010/Ethanol/TPGS | 138 | Poor | Slow |
| 61/8/25/5 wt % E681010/Ethanol/TPGS/Labrafil M2125 | 167 | N/A | N/A |
| 66/8/21/5 wt % E681010/Ethanol/TPGS/Labrafil M2125 | 162 | Partial | Slow |
| 68/10/13/10 wt % E681010/Ethanol/TPGS/Poloxamer331 | 162 | None | N/A |
| 68/13/10/9 wt % E681010/EthanoI/TPGS/Span80 | 158 | Poor | Slow |
| 67/13/16/5 wt % E681010/Ethanol/TPGS/Labrafil M2125 | 174 | N/A | N/A |
| 68/12/15/5 wt % E681010/Ethanol/TPGS/ | 157 | Poor | Slow |
| Labrafil M1944CS | | | |
| 67/13/16/5 wt % E681010/Ethanol/TPGS/ | 175 | Partial | Fast |
| Labrafil M2125CS | | | |
| 66/13/16/4 wt % E681010/Ethanol/TPGS/Span80 | 157 | Poor | Slow |
| 67/13/17/4 wt % E681010/EthanoI/TPGS/Span85 | 161 | Poor | Fast |
| 66/13/19/3 wt % E681010/Ethanol/TPGS/ | 175 | Poor | Fast to Slow |
| Labrafil M2125CS | | | |
| 65/22/12 wt % E681010/Ethanol/Tween80 | 170 | Poor | Fast |
| 77/11/13 wt % E681010/Ethanol/Tween80 | 176 | None | N/A |
| 72/5/11/12 wt % E681010/Ethanol/Tween80/Span85 | 167 | None | N/A |
| 66/21/12/ wt % E681010/Ethanol/Tween85 | 165 | Poor | Fast |
| 77/10/13 wt % E681010/Ethanol/Tween85 | 176 | None | N/A |

### Example 18

### Surfactant-Containing Fenofibrate Formulations

Two formulations were prepared for the administration of 145 mg of fenofibrate. Formulation A contained 145 mg fenofibrate in an 800 microliter capsule (91 mg/mL fenofibrate).

TABLE 22
Formulation A

| | | Per dose (mg)- |
|---|---|---|
| Component | Weight Percent | (2 capsules) |
| Fenofibrate | 10.6 | 145 |
| E681010 | 58.1 | 794 |
| Ethanol | 13.4 | 183 |
| Cremophor EL | 8.9 | 122 |
| Span 20 | 8.9 | 122 |

Formulation B contained 145 mg fenofibrate in an 650 microliter capsule (111 mg/mL fenofibrate).

TABLE 23
Formulation B

| | | Per dose (mg)- |
|---|---|---|
| Component | Weight Percent | (2 capsules) |
| Fenofibrate | 12.6 | 145 |
| E68101O | 56.8 | 656 |
| Ethanol | 13.2 | 152 |
| Cremophor EL | 8.7 | 101 |
| Span 20 | 8.7 | 101 |

Two formulations were also prepared for the administration of 130 mg of fenofibrate. Formulation C contained 130 mg fenofibrate in an 800 microliter capsule (81 mg/mL fenofibrate).

TABLE 24
Formulation C

| | | Per dose (mg)- |
|---|---|---|
| Component | Weight Percent | (2 capsules) |
| Fenofibrate | 9.6 | 130 |
| E681010 | 58.8 | 800 |
| Ethanol | 13.6 | 185 |
| Cremophor EL | 9.0 | 123 |
| Span 20 | 9.0 | 123 |

Formulation D contained 130 mg fenofibrate in an 650 microliter capsule (100 mg/mL fenofibrate).

TABLE 25
Formulation D

| | | Per dose (mg)- |
|---|---|---|
| Component | Weight Percent | (2 capsules) |
| Fenofibrate | 11.5 | 130 |
| E681010 | 57.5 | 648 |
| Ethanol | 13.2 | 149 |
| Cremophor EL | 8.9 | 100 |
| Span 20 | 8.9 | 100 |

### Example 19

### Physical Stability Characterization of Fenofibrate in Various Oils at 15 Degrees C.

Fenofibrate solutions were prepared at a concentration of 65 mg/mL in pure oil and mixtures of oil and ethanol at room temperature. The solutions were incubated at 15 degrees C. and periodically observed for precipitation of fenofibrate.

Table 26 shows results of visual observation of oil samples with 13 percent w/w ethanol after 18 days at 15 degrees C.

TABLE 26
Physical Stability of Fenofibrate in Mixtures of Oil and Ethanol

| Oil with 13 wt % Ethanol | 18-day observation |
|---|---|
| Myvacet | Clear solution |
| Epax 1050TG | Clear solution |
| Epax 4510TG | Clear solution |
| Cod Liver Oil | Clear solution |
| E681010 | Clear solution |

Table 27 shows results of visual observation of pure oil samples after 18 days at 15 degrees C.

TABLE 27
Physical Stability of Fenofibrate in Pure Oil

| Oil | 18-day observation |
|---|---|
| Myvacet | Clear solution |
| Epax 1050TG | Precipitation |
| Epax 4510TG | Precipitation |
| Cod Liver Oil | Precipitation* |
| E681010 | Clear solution |

*= This sample did not form a 65 mg/mL solution at room temperature.

As suggested in the data above, ethanol appears to enhance the physical stability of fenofibrate solubilized in some oils.

### Example 20

### Fenofibrate Formulations

The following formulations comprise fenofibrate in about 145 mg doses, where two capsules are administered per dose. Table 28 describes several embodiments of non-surfactant-containing fenofibrate formulations.

TABLE 28
Fenofibrate formulations without surfactant

| | 80 | 90 | 100 |
|---|---|---|---|
| Fenofibrate Concentration (mg/mL) | | | |
| Formulation Density (g/mL) | 0.912 | 0.918 | 0.914 |
| Dose Volume (mL) | 1.81 | 1.61 | 1.45 |
| Capsule Volume (mL) | 0.907 | 0.805 | 0.725 |
| E681010 dose (g) | 1.31 | 1.16 | 1.02 |
| DHA and EPA content (g) | 1.02 | 0.90 | 0.80 |
| Total Omega-3 content (g) | 1.14 | 1.01 | 0.89 |
| Composition- Mass Percent | | | |
| Fenofibrate | 8.8 | 9.8 | 10.9 |
| E681010 | 79.2 | 78.3 | 77.3 |
| Ethanol | 12.0 | 11.9 | 11.8 |

Table 29 describes several embodiments of surfactant-containing fenofibrate formulations.

TABLE 29
Fenofibrate formulations comprising surfactant

| | 70 | 80 | 90 |
|---|---|---|---|
| Fenofibrate Concentration (mg/mL) | | | |
| Formulation Density (g/mL) | 0.938 | 0.935 | 0.943 |
| Dose Volume (mL) | 2.07 | 1.81 | 1.61 |
| Capsule Volume (mL) | 1.033 | 0.906 | 0.806 |
| E681010 dose (g) | 1.17 | 1.01 | 0.89 |
| DHA and EPA content (g) | 0.91 | 0.78 | 0.69 |
| Total Omega-3 content (g) | 1.02 | 0.88 | 0.78 |
| Composition- Mass Percent | | | |
| Fenofibrate | 7.5 | 8.6 | 9.5 |
| E681010 | 60.1 | 59.4 | 58.8 |
| Ethanol | 13.9 | 13.7 | 13.6 |
| Cremophor EL | 9.2 | 9.1 | 9.0 |
| Span 20 | 9.3 | 9.2 | 9.1 |

Table 30 describes a fenofibrate formulation where the solubility of fenofibrate is 106 mg/mL at 15 degrees C. The actual fenofibrate concentration in the formulation is 90.6 mg/mL. A single dose of this formulation (two capsules) includes 0.83 grams of omega-3 oil. This formulation provides similar emulsification to that observed in a similar formulation with a greater percentage of ethanol (13.6 weight percent).

TABLE 30
Fenofibrate formulation comprising surfactant

| Component | Weight Percent | Per Dose (mg) (2 capsules) |
|---|---|---|
| Fenofibrate | 9.6 | 145.0 |
| E681010 | 63.4 | 957.6 |
| Ethanol | 9.0 | 135.9 |
| Cremophor EL | 9.0 | 135.9 |
| Span 20 | 9.0 | 135.9 |

### Example 21

### Solubility Studies at 4 and 15 Degrees C.

Table 31 includes fenofibrate solubility data of four liquid formulations at 4 and 15 degrees C.

TABLE 31
Fenofibrate solubility in four formulations at 4 and 15 degrees C.

| | Formulation Composition (weight percent) | | | | Solubility (mg/mL) | |
|---|---|---|---|---|---|---|
| Formulation | E681010 | Ethanol | Cremophor EL | Span 20 | 4 deg C. | 15 deg C. |
| E | 65.0 | 15.0 | 10.0 | 10.0 | 70 | 115 |
| F | 70.0 | 10.0 | 10.0 | 10.0 | 72 | 107 |
| G | 64.9 | 10.0 | 12.5 | 12.6 | 70 | 95 |
| H | 65.0 | 8.5 | 13.3 | 13.3 | 68 | 94 |

### Example 22

### Physical Stability Characterization

Table 32 includes precipitation and resolubilization times for two fenofibrate formulations. For this study, both formulations were incubated at 4 degrees C. and observed for precipitation of fenofibrate. Both formulations J and K precipitated fenofibrate after 2 days. Following such precipitation, the formulations were brought to room temperature and the duration for resolubilization was observed. Formulation J took 2 days to resolubilize at room temperature while formulation K took at least 7 days to resolubilize. Formulations J and K were also incubated at 15 degrees C. and did not precipitate after 14 days.

TABLE 32
Physical stability study of fenofibrate in two formulations

| | Formulation Composition (weight percent) | | | | |
|---|---|---|---|---|---|
| | | | | Cremophor | |
| Formulation | E681010 | Ethanol | Fenofibrate | EL | Span 20 |
| J | 58.9 | 13.6 | 9.3 | 9.1 | 9.1 |
| K | 63.3 | 9.0 | 9.6 | 9.0 | 9.0 |

1-37. (canceled)

38. A liquid formulation comprising fenofibrate dissolved in a vehicle comprising an omega-3 ester or omega-3 alkyl ester and an alcohol, wherein: the formulation comprises (i) about 5% to about 20% by weight of fenofibrate; (ii) about 55% to about 85% by weight of an omega-3 ester or omega-3 alkyl ester; (iii) about 5% to about 20% by weight of an alcohol; and (iv) about 5% to about 25% by weight of a surfactant.

39. The liquid formulation of claim 38, wherein the solubility of the fenofibrate in the vehicle is about 50 mg/mL to about 200 mg/mL.

40. The liquid formulation of claim 38, wherein said omega-3 ester or omega-3 alkyl ester comprises EPA and DHA in an amount which is between about 70 and about 90 percent by weight of the total weight of said omega-3 ester or omega-3 alkyl ester.

41. The liquid formulation of claim 38, wherein said omega-3 ester or omega-3 alkyl ester comprises EPA and DHA, and the EPA:DHA ratio is from about 3:1 to about 1:1.

42. The liquid formulation of claim 38, wherein said omega-3 ester or omega-3 alkyl ester comprises EPA and DHA, and the EPA:DHA ratio is from about 10:1 to about 5:1.

43. A method of increasing the solubility of fenofibrate in an omega-3 oil, comprising adding an alcohol to said omega-3 oil.

44. The method of claim 43, wherein said solubility of fenofibrate is increased by at least about 50 percent.

45. The method of claim 43, wherein said omega-3 oil is an omega-3 ethyl ester.

46. The method of claim 43, wherein said alcohol is ethanol.

47. The method of claim 43, wherein said alcohol comprises from about 5 percent to about 20 percent by weight of the total formulation.

48. A liquid formulation comprising fenofibrate and omega-3 oil, wherein the omega-3 oil is an omega-3 ethyl ester.

49. The liquid formulation of claim 48, wherein the concentration of fenofibrate is between about 60 mg/mL and about 170 mg/mL.

50. The liquid formulation of claim 48, wherein the concentration of fenofibrate is between about 70 mg/mL and about 140 mg/mL.

51. The liquid formulation of claim 48, wherein the concentration of fenofibrate is between about 75 mg/mL and about 100 mg/mL.

52. The liquid formulation of claim 48, wherein said liquid formulation is chemically stable at about 40 degrees C. for 8 weeks.

53. The liquid formulation of claim 48, wherein said liquid formulation is physically stable at about 15 degrees C. for 2 weeks.

54. A liquid formulation comprising fenofibrate, omega-3 oil, and ethanol.

55. The liquid formulation of claim 54, wherein the omega-3 oil is an omega-3 ethyl ester.

56. The liquid formulation of claim 54, wherein the concentration of fenofibrate is between about 60 mg/mL and about 170 mg/mL.

57. The liquid formulation of claim 54, wherein the concentration of fenofibrate is between about 70 mg/mL and about 140 mg/mL.

58. The liquid formulation of claim 54, wherein the concentration of fenofibrate is between about 75 mg/mL and about 100 mg/mL.

59. The liquid formulation of claim 54, wherein said liquid formulation is chemically stable at about 40 degrees C. for 8 weeks.

60. The liquid formulation of claim 54, wherein said liquid formulation is physically stable at about 15 degrees C. for 2 weeks.

61. The liquid formulation of claim 54, further comprising a surfactant.

62. The liquid formulation of claim 61, wherein said surfactant comprises less than about 20% by weight of said liquid formulation.

63. The liquid formulation of claim 54, wherein the formulation does not contain a surfactant.

64. A liquid formulation comprising about 55 to about 85% by weight of an omega-3 ester oil, about 5 to 20% by weight of a C₁ to C₄ alcohol, and fenofibrate, wherein the fenofibrate concentration is from about 60 to 170 mg/mL at 25 degrees C.

65. The liquid formulation of claim 64, wherein the fenofibrate concentration is from about 60 to about 120 mg/mL at 25 degrees C.

66. The liquid formulation of claim 64, wherein the fenofibrate concentration is from about 60 to about 100 mg/mL at 25 degrees C.

67. A liquid formulation comprising about 65 to 90% by weight of an omega-3 ester oil and fenofibrate, wherein the fenofibrate concentration is from about 100 to about 140 mg/mL at 25 degrees C.

68. The liquid formulation of claim 67, wherein the fenofibrate concentration is from about 100 to about 120 mg/mL at 25 degrees C.

69. The liquid formulation of claim 67, wherein the fenofibrate concentration is from about 120 to about 140 mg/mL at 25 degrees C.

70. Fenofibrate polymorph Form II.

71. The fenofibrate polymorph Form II of claim 70, wherein said polymorph exhibits a powder X-ray diffraction pattern comprising peaks at 12.51, 15.43, and 19.13 degrees 2-theta.

72. The fenofibrate polymorph Form II of claim 70, wherein said polymorph exhibits a powder X-ray diffraction pattern substantially as shown in FIG. 8.

73. A liquid formulation comprising fenofibrate dissolved in a vehicle comprising an omega-3 ester or omega-3 alkyl ester and an alcohol, wherein:
(a) the formulation comprises (i) about 5% to about 20% by weight of fenofibrate (ii) about 55% to about 85% by weight of an omega-3 ester or omega-3 alkyl ester, and (iii) about 5% to about 20% by weight of an alcohol;
(b) the solubility of the fenofibrate in the vehicle is from about 50 mg/mL to about 200 mg/mL at 25 degrees C.; and
(c) the liquid formulation does not contain a surfactant.

74. A method of treating a patient suffering from hyperlipidemia comprising administering to the patient a therapeutically effective amount of a liquid formulation of claim 38.

75. A method of treating a patient suffering from hyperlipidemia comprising administering to the patient a therapeutically effective amount of a liquid formulation of claim 48.

76. A method of treating a patient suffering from hyperlipidemia or hypertriglyceridemia comprising administering to the patient a therapeutically effective amount of a liquid formulation comprising fenofibrate and omega-3 oil.
